(19) United States
(12) Patent Application Publication
(10) Pub. No.: US 2003/0158216 A1
(43) Pub. Date: Aug. 21, 2003

(54) 2-acyl indole derivatives and their use as antitumor agents

(75) Inventors: Thomas Beckers, Frankfurt am Main (DE); Silke Bassner, Schoneck (DE); Thomas Klenner, Ingelheim (DE); Siavosh Mahboobi, Regensburg (DE); Herwig Pongratz, Regensburg (DE); Markus Frieser, Maxhutte-Rappenbugl (DE); Harald Hufsky, Galmersheim (DE); Jorg Hockemeyer, Bremen (DE); Heinz-Herbert Fiebig, Freiburg (DE); Angelika Burger, Freiburg (DE); Frank-D. Bohmer, Dorndorf (DE)

Correspondence Address: PILLSBURY WINTHROP, LLP, P.O. BOX 10500, MCLEAN, VA, 22102, US

(73) Assignee: Baxter Oncology GmbH

(21) Appl. No.: 10/279,123

(22) Filed: Oct. 24, 2002

Related U.S. Application Data
(63) Continuation of application No. 09/843,139, filed on Apr. 27, 2001, now abandoned.

(30) Foreign Application Priority Data
Apr. 28, 2000 (DE) 100 20 852.5
Jan. 20, 2001 (DE) 101 02 629.3

(51) Int. Cl. C07D 491/02; C07D 471/02; A61K 31/519; A61K 31/4745

(52) U.S. Cl. 514/265.1; 514/300; 544/280; 546/113

(57) ABSTRACT

The invention relates to novel indole and heteroindole derivatives of the formula I

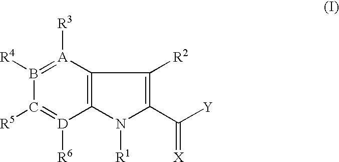

to their tautomers, their stereoisomers, their mixtures and their salts, to their preparation and to the use of indole derivatives of the formula I as antitumor agents.

The invention relates to novel indole and heteroindole derivatives of the formula I

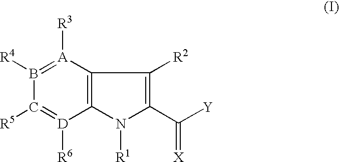

to their tautomers, their stereoisomers, their mixtures and their salts, to their preparation and to the use of indole derivatives of the formula I as antitumor agents.

It is an object of the present invention to provide novel active compounds for the treatment of tumors in mammals.

The German Offenlegungsschrift [German published specification] No. DE 2 501 468 describes 1-alkyl-2-pyridylcarbonyl-substituted indole compounds, their preparation and their use as fibrinolytics or thrombolytics. An antitumor action is neither described nor suggested.

In the Belgian patent No. BE 637355, 2-benzoyl-substituted indole compounds as intermediates in a Grignard reaction are converted into the corresponding 1-aminoalkyl-1-hydroxy derivatives (phenylindolyl-alkanolamines). A biological action of the intermediates is neither described nor suggested to a person of ordinary skill in the art.

The German Offenlegungsschrift No. DE 2 037 998 describes a process for preparing 2-benzoyl-, 2-acetyl, 2-propionyl and 2-p-toluoylindole, the class of the 2-acylindoles being described as “relatively inaccessible”. Reference is made to the use of the 2-acylindoles as intermediates in the preparation of phenylindolylalkanolamine sedatives according to the abovementioned Belgian patent No. 637 355. Without further details being given, the use of the 2-acylindoles for preparing dyes, alkaloids, plant hormones and proteins is merely mentioned. A use of the 2-acylindoles as medicaments is neither disclosed nor suggested.

In the publication with the title “Nucleophilic Substitution of C-Hydrogen on the Five-membered Ring of Indoles” by John A. Joule in Progress in Heterocyclic Chemistry, 86VK, 7200.6-11, pages 45-65, the preparation of hydroxy-2-indolyl-(2-hydroxymethyl)-phenylmethane is described on page 50, the preparation of 2-benzoylindole is described on page 54 and the preparation of 2-cyclopropycarbonylindole is described on page 55. A medicinal use of the compounds mentioned is neither disclosed nor suggested.

The publication by David St. C. Black et al., J. Chem. Soc., Chem. Commun., 1989, pp. 425-426 describes the preparation of 2-(p-chlorophenylcarbonyl-)-3-methyl-4,6-dimethoxyindole and its use as an intermediate in the synthesis of indole-containing macrocycles.

U.S. Pat. No. 3,660,430 by Meier E. Freed et al., granted on 2 May 19972 [sic], describes 3-phenyl-substituted 2-benzoylindole compounds, their reparation and their use as CNS sedatives.

U.S. Pat. No. 3,838,167 by Charles D. Jones, granted on 24 Sep. 1974, describes a process for preparing 2-acylindole compounds. The only example given for a 2-benzoylindole that is unsubstituted in the 3-position is 2-(3-bromobenzoyl)-7-trifluoromethylindole. With respect to the use as CNS sedative, reference is made to the abovementioned U.S. Pat. No. 3,660,430.

The publication by Michael D. Varney et al., J. Med. Chem. 1994, 37, pages 2274-2284, describes 2-benzoyl-(metaposition: H, trifluoromethyl or methyl) and 2-cyclohexylcarbonyl indole compounds as intermediates for the preparation of HIV protease inhibitors. A biological action of the intermediates is neither disclosed nor suggested.

The publication by Gordon W. Gribble et al., J. Org. Chem. 1992, 57, 5891-5899 describes 2-(2-carboxy)-benzoyl and 2-(5-carboxypyridin-4-yl indole derivatives, the latter being substituted in the 5-position by hydrogen or methoxy, as intermediates for the synthesis of benzo[b]carbazole and 6H-pyrido-[4,3-b]carbazoles respectively. A biological action of the intermediates is neither disclosed nor suggested.

The publication by S. Cenini, Journal of Molecular Catalysis A: Chemical 111 (1996) 37-41 describes the palladium- or ruthenium-catalyzed synthesis of 2-benzoylindoles which are unsubstituted in the indole ring, where the phenyl ring is substituted in positions 3, 4 or 5 by hydrogen, halogen, methyl or methoxy. A biological action of the 2-acylindoles that are prepared is not disclosed.

The publication by David St. C. Black and L. C. H. Wong, J.C.S. Comm. 1980, page 200, describes the synthesis of 2-acylindoles which are substituted in indole positions 4 to 7 by chlorine, methyl or methoxy. A biological action of the 2-acylindoles that are prepared is neither disclosed nor suggested.

The publication by David St. C. Black et al., Tetrahedron Letters, Vol. 32, No. 12, pages 1587-1590, 1991 describes the reaction of 3-methyl-4,7-dimethoxy-2-benzoylindole with methyl iodide with formation of the corresponding carbinol compound. A biological action of the starting material is neither disclosed nor suggested.

The publication by Tetsuji Kametani et al., Yakugaku-zasshi, 91 (9) 1033-1036 (1971) describes a process for preparing the compound 2-benzoyl-5,6-methylenedioxy-indole from β-(benzoyl)-4,5-methylenedioxy-2-nitro-styrene.

The publication by Charles D. Jones and Tulio Suarez, J. Org. Chem., Vol. 37, No. 23, 1972, pages 3622-3623 describes a process for preparing 2-acylindoles. A biological action of the compounds that are prepared is neither disclosed nor suggested.

The publication by V. I. Gorgos et al., Khimiya Geterotsiklicheskikh Soedinenii, No. 11, pp. 1490-1492 (English translation in UDC 547.756′757-07; pp. 1179-1182) describes a process for preparing 2-benzoyl-indoles substituted in the 5- or 7-position by bromine or methoxy. A biological action of the compounds that are prepared is not disclosed The same applies to the Soviet patent No. 696016, which names the authors of the publication mentioned above as inventors.

Surprisingly, it has now been found that the compounds of the formula I

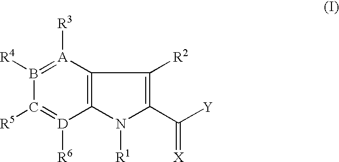

in which

R1 is hydrogen, (C1-C6)-alkylcarbonyl, preferably acetyl, (C₁-C₆)-alkyl, mono-(C₁-C₆)-alkylamino-(C₁-C₄)-alkyl, di-(C₁-C₆)-alkylamino-(C₁-C₄)-alkyl, where the two (C₁-C₆)-alkyl radicals together may form a ring, which optionally contains one or more NH, N-(C1-C6)-alkyl, O or S members, (C6-C14)-aryl-(C1-C6)-alkyl or (C6-C14)-aryl-(C1-C6)-alkoxy-(C1-C6)-alkyl;

R2 is a hydrogen atom, halogen, cyano, nitro, (C1-C6)-alkyl, (C1-C6)-alkyl which is substituted by one or more halogen atoms, preferably trifluoromethyl, (C1-C6)-alkoxy which is substituted by one or more halogen atoms, preferably trifluoromethoxy, (C2-C6)-alkenyl, (C2-C6)-alkynyl, (C3-C8)-cycloalkyl, (C1-C6)-alkoxy, (C1-C6)-alkoxycarbonyloxy, (C1-C6)-alkylcarbonyloxy, (C1-C4)-alkylthio, (C1-C4)-alkylsulfinyl, (C1-C4)-alkylsulfonyl, (C1-C6)-alkoxy-(C1-C6)-alkyl, amino, mono-(C1-C6)-alkylamino, di-N,N-(C1-C6)-alkylamino, where the two (C1-C6)-alkyl radicals together may form a ring, which optionally contains one or more NH, N-(C1-C6)-alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkylcarbonyl, (C1-C6)-alkoxycarbonyl or hydroxyl;

A, B, C and D independently of one another are a nitrogen atom (in which case R3, R4, R5 and R6 represent the free electron pair at the nitrogen atom) or are a carbon atom substituted by one of the radicals R3-R6;

R3, R4, R5 and R6 independently of one another are, when attached to nitrogen, a free electron pair, or, when attached to carbon, hydrogen, halogen, cyano, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, (C2-C6)-alkenyl, (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, (C1-C6)-alkoxycarbonyl, (C1-C6)-alkoxycarbonyloxy, (C1-C6)-alkylcarbonyl, (C1-C6)-alkylcarbonyloxy, (C1-C4)-alkylthio, (C1-C4)-alkylsulfinyl, (C1-C4)-alkylsulfonyl, carboxyl, (C1-C6)-alkyl carboxylate, carboxamide, N-(C1-C4)-alkyl-carboxamide, N,N-di-(C1-C4)-alkyl-carb-oxamide, (C1-C6)-alkoxy-(C1-C6)-alkyl, amino, mono-(C1-C6)-alkylamino, N,N-di-(C1-C6)-alkylamino, where the two C1-C6-alkyl radicals together may form a ring, which optionally contains one or more NH, N-(C1-C6)-alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkylcarbonyl, (C1-C6)-alkoxycarbonyl, hydroxyl, where two directly adjacent radicals may be attached to one another;

Y is unsubstituted (C6-C14)-aryl or (C6-C14)-aryl which is fully or partially substituted by identical or different substituents, preferably phenyl or 1- or 2-naphthyl, or is unsubstituted (C1-C13)-heteroaryl or (C1-C13)-heteroaryl which is fully or partially substituted by identical or different substituents and has in each case at least one to four N, NH, N-(C1-C6)-alkyl, O and/or S as ring members, or is unsubstituted (C3-C8)-cycloalkyl or (C3-C8)-cycloalkyl which is fully or partially substituted by identical or different substituents, where the identical or different substituents are selected independently of one another from the group consisting of halogen, preferably fluorine, chlorine, bromine or iodine; cyano; straight-chain or branched cyano-(C1-C6)-alkyl; hydroxyl; straight-chain or branched (C1-C6)-alkyl which is substituted by one or more hydroxyl substituents; carboxyl; (C1-C6)-alkyl carboxylate, carboxamide; N-(C1-C6)-alkyl-carboxamide, N,N-di-(C1-C4)-alkyl-carboxamide, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, straight-chain or branched (C2-C6)-alkenyl, straight-chain or branched (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, thio (—SH), straight-chain or branched (C1-C6)-alkylthio, (C1-C6)-alkylsulfinyl, (C1-C6)-alkyl-sulfonyl, (C1-C6)-alkoxy-(C1-C6)-alkyl, amino, straight-chain or branched mono-(C1-C6)-alkylamino, straight-chain or branched N,N-di-(C1-C6)-alkylamino, where the two (C1-C6)-alkyl radicals together may form a ring, which may optionally contain one or more NH, N-(C1-C6)-alkyl, O and/or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkyl-carbonyl, (C1-C6)-alkylcarbonyloxy, (C1-C6)-alkoxycarbonyl, (C1-C6)-alkoxycarbonyloxy, straight-chain or branched mono- and N,N-di-(C1-C6)-alkylcarbonylamino, straight-chain or branched is mono- and N,N-di-(C1-C6)-alkoxycarbonylamino, straight-chain or branched N-(C1-C6)-alkylcarbonyl-N-(C1-C6)-alkylamino, straight-chain or branched N-(C1-C6)-alkoxycarbonyl-N-(C1-C6)-alkylamino, formylamino, formyl, where two directly adjacent radicals may be attached to one another;

X is an oxygen or sulfur atom, is NH, or is a geminally (at the same C atom) substituted hydroxyl and hydrogen (—CH(OH)—);

their stereoisomers, their tautomers, mixtures thereof and the pharmaceutically acceptable salts thereof are suitable for preparing a medicament for the treatment of oncoses in mammals.

According to a further aspect of the invention, compounds of the formula I according to claim 1 are used for preparing an antitumor agent, in which compounds R1-R6, A, B, C, D, X and Y are as defined above, with the proviso that at least one of the radicals R3-R6 is straight-chain or branched (C1-C6)-alkoxy, preferably methoxy; straight-chain or branched (C1-C6)-alkyl, preferably methyl; straight-chain or branched (C1-C6)-alkylenedioxy, preferably methylene-dioxy, hydroxyl; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl; hydroxyl; (C1-C6)-alkylcarbonyloxy, (C1-C6)-alkoxycarbonyloxy.

According to a further aspect of the invention, compounds of the formula I according to claim 1 are used for preparing an antitumor agent, in which compounds R1, R2, R3, R5, R6, A, B, C, D, X and Y are as defined above, with the proviso that the radical R4 is straight-chain or branched (C1-C6)-alkoxy, preferably methoxy; straight-chain or branched (C1-C6)-alkyl, preferably methyl; straight-chain or branched (C1-C6)-alkylenedioxy (where the second oxygen atom may optionally be the radical R4 or R6), preferably methylenedioxy, hydroxyl; (C1-C6)-alkylcarbonyloxy, (C1-C6)-alkoxycarbonyloxy; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

According to a further aspect of the invention, compounds of the formula I according to claim 1 are used for preparing an antitumor agent, in which compounds R1, R2, R3, R5, R6, A, B, C, D, X and Y are as defined above, with the proviso that the radical R4 is straight-chain or branched (C1-C6)-alkoxy, preferably methoxy.

According to a further aspect of the invention, compounds of the formula I according to claim 1 are used for preparing an antitumor agent, in which compounds R1, R2, R3, R5, R6, A, B, C, D, X and Y are as defined above, with the proviso that the radical R4 is methoxy.

According to a further aspect of the invention, compounds of the formula I according to claim 1 are used for preparing an antitumor agent, in which compounds R1-R6, A, B, C, D and X are as defined above, with the proviso that the radical Y is (C6-C14)-aryl or (C1-C13)-heteroaryl which contains at least one N, NH, O and/or S as ring members, which is substituted by at least one radical selected from the group consisting of hydrogen, amino, halogen, nitro, cyano, straight-chain or branched (C1-C6)-alkoxy, preferably methoxy; straight-chain or branched (C1-C6)-alkyl, preferably methyl; hydroxyl; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

According to a further aspect of the invention, compounds of the formula I according to claim 1 are used for preparing an antitumor agent, in which compounds R1-R6, A, B, C, D and X are as defined above, with the proviso that the radical Y is substituted or unsubstituted (C6-C14)-aryl or (C1-C13)-heteroaryl which contains at least one to four N, NH, O and/or S as ring members.

According to a further aspect of the invention, compounds of the formula I according to claim 1 are used for preparing an antitumor agent, in which compounds R1-R6, A, B, C, D and X are as defined above, and the radical Y is (C6-C14)-aryl or (C1-C13)-heteroaryl which contains at least one N, NH, O and/or S as ring members, which is substituted by at least one radical selected from the group consisting of hydrogen, amino, halogen, nitro, cyano, straight-chain or branched (C1-C6)-alkoxy, preferably methoxy; straight-chain or branched (C1-C6)-alkyl, preferably methyl; hydroxyl; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

According to a further aspect of the invention, compounds of the formula I according to claim 1 are used for preparing an antitumor agent, in which compounds R1-R6, A, B, C, D and X are as defined above, and the radical Y is a 1-phenyl radical which is unsubstituted or substituted by hydrogen, 3,4-dichloro, 2- or 3-methoxy, 2,4-dimethoxy, 3-nitro 3-trifluoromethyl, 2,3,4-trimethoxy, 3,4,5-trimethoxy.

According to a further aspect of the invention, novel compounds of the formula I

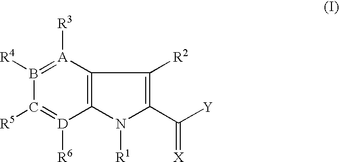

in which

R1 is hydrogen, (C1-C6)-alkylcarbonyl, preferably acetyl, (C₁-C₆)-alkyl, mono-(C₁-C₆)-alkylamino-(C₁-C₄)-alkyl, N,N-di(C₁-C₆)-amino-(C₁-C4)-alkyl, where the two (C₁-C₄)-alkyl radicals together may form a ring, which optionally contains one or more NH, N-(C1-C[lacuna])-alkyl, O or S members, (C6-C14)-aryl-(C1-C6)-alkyl or (C6-C14)-aryl-(C1-C6)-alkoxy-(C1-C6)-alkyl;

R2 is a hydrogen atom, halogen, cyano, nitro, (C1-C6)-alkyl, (C1-C6)-alkyl which is substituted by one or more halogen atoms, preferably trifluoromethyl, (C1-C6)-alkoxy which is substituted by one or more halogen atoms, preferably trifluoromethoxy, (C2-C6)-alkenyl, (C2-C6)-alkynyl, (C3-C8)-cycloalkyl, (C1-C[lacuna])-alkoxy, (C1-C[lacuna])-alkoxycarbonyloxy, (C1-C[lacuna])-alkylcarbonyloxy, (C1-C[lacuna])-alkylthio, (C1-C4)-alkylsulfinyl, (C1-C4)-alkylsulfonyl, (C1-C4)-alkoxy-(C1-C4)-alkyl, amino, mono-(C1-C[lacuna])-alkylamino, di-(C1-C[lacuna])-alkyl)-amino, where the two C1-C4-alkyl radicals together may form a ring, which optionally contains one or more NH, N-(C1-C4)alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkyl-carbonyl, (C1-C6)-alkoxycarbonyl or hydroxyl;

A, B, C and D independently of one another are a nitrogen atom (in which case R3, R4, R5 and R6 represent the free electron pair at the nitrogen atom) or are a carbon atom substituted by one of the radicals R3-R6;

R3, R4, R5 and R6 independently of one another are, when attached to nitrogen, a free electron pair, or, when attached to carbon, hydrogen, halogen, cyano, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, (C2-C6)-alkenyl, (C2-C6)-alkynyl, (C3-C8)-cycloalkyl, straight-chain or branched (C1-C6)-alkoxy, straight-chain or branched (C1-C6)-alkylenedioxy, (C1-C6)-alkoxycarbonyloxy, (C1-C6)-alkylcarbonyloxy, (C1-C[lacuna])-alkylthio, (C1-C4)-alkylsulfinyl, (C1-C4)-alkylsulfonyl, carboxyl, (C1-C6)-alkyl carboxylate, carboxamide, N-(C1-C4)-alkyl-carboxamide, N,N-di-(C1-C4)-alkyl-carboxamide, (C1-C6)-alkoxy-(C1-C6)-alkyl, amino, mono-(C1-C6)-alkylamino, di-(C1-C6)-alkyl)-amino, where the two C1-C4-alkyl radicals together may form a ring, which optionally contains one or more NH, N-(C1-C4)-alkyl, O or S, aryl, aryloxy, aryl-(C1-C4)-alkyl, aryl-(C1-C4)-alkoxy-(C1-C4)-alkyl, (C1-C6)-alkylcarbonyl, (C1-C6)-alkoxycarbonyl, hydroxyl, where two directly adjacent radicals may be attached to one another;

Y is unsubstituted (C10-C14)-aryl or (C10-C14)-aryl which is fully or partially substituted by identical or different substituents, preferably 1- or 2-naphthyl, or is unsubstituted (C1-C13)-heteroaryl or (C1-C13)-heteroaryl which is fully or partially substituted by identical or different substituents and has in each case at least one to four N, NH, N-(C1-C6)-alkyl, O and/or S as ring members, or is unsubstituted (C3-C8)-cycloalkyl or (C3-C8)-cycloalkyl which is fully or partially substituted by identical or different substituents, where the identical or different substituents are selected independently of one another from the group consisting of halogen, preferably fluorine, chlorine, bromine or iodine; cyano; straight-chain or branched cyano-(C1-C6)-alkyl; hydroxyl; straight-chain or branched (C1-C6)-alkyl which is substituted by one or more hydroxyl groups; carboxyl; (C1-C6)-alkyl carboxylate; carboxamide; N-(C1-C6)-alkyl-carboxamide, N,N-di-(C1-C4)-alkyl-carboxamide, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, straight-chain or branched (C2-C6)-alkenyl, straight-chain or branched (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, thio (—SH), straight-chain or branched (C1-C6)-alkylthio, (C1-C6)-alkylsulfinyl, (C1-C6)-alkylsulfonyl, (C1-C6)-alkoxy-(C1-C6)-alkyl, amino, straight-chain or branched mono-(C1-C6)-alkylamino, straight-chain or branched N,N-di-(C1-C6)-alkylamino, where the two (C1-C6)-alkyl radicals together may form a ring, which may optionally contain one or more NH, N-(C1-C6)-alkyl, O and/or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkoxycarbonyl, straight-chain or branched mono- and N,N-di-(C1-C6)-alkylcarbonylamino, straight-chain or branched mono-N- and N,N-di-(C1-C6)-alkoxycarbonylamino, straight-chain or branched N-(C1-C6)-alkylcarbonyl-N-(C1-C6)-alkylamino, straight-chain or branched N-(C1-C6)-alkoxycarbonyl-N-(C1-C6)-alkylamino, formylamino, formyl, where two directly adjacent radicals may be attached to one another;

X is an oxygen or sulfur atom, is NH, or is a generally (at the same C atom) substituted hydroxyl and hydrogen (—CH(OH)—);

their stereoisomers, their tautomers, and the pharmaceutically acceptable salts thereof, except for the racemic compounds according to formula I where R1=R2=R3=R5=R6=hydrogen, X=oxygen or, if R4=H, generally substituted hyroxyl [sic] and hydrogen, Y=3-carboxypyridin-4-yl and R4=hydrogen or methoxy, and the compounds 2-cyclopropylcarbonylindole and 2-cyclohexylcarbonylindole, are provided

According to a further aspect of the invention, compounds of the formula I

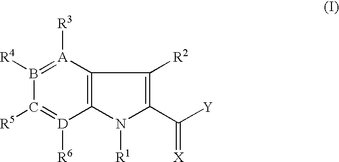

in which

R1 is hydrogen, (C1-C6)-alkylcarbonyl, preferably acetyl, (C₁-C₆)-alkyl, mono-(C₁-C₆)-alkylamino-(C₁-C₄)-alkyl, di(C₁-C₆)-(lacuna]amino-(C₁-C₄)-alkyl, where the two (C₁-C₄)-alkyl radicals together may form a ring, which optionally contains one or more NH, N-(C1-C[lacuna])-alkyl, O or S members, (C6-C14)-aryl-(C1-C6)-alkyl or (C6-C14)-aryl-(C1-C6)-alkoxy-(C1-C6)-alkyl;

R2 is a hydrogen atom, halogen, cyano, nitro, (C1-C6)-alkyl, (C1-C6)-alkyl which is substituted by one or more halogen atoms, preferably trifluoromethyl, (C1-C6)-alkoxy which is substituted by one or more halogen atoms, preferably trifluoromethoxy, (C2-C6)-alkenyl, (C2-C6)-alkynyl, (C3-C8)-cycloalkyl, (C1-C[lacuna])-alkoxy, (C1-C[lacuna])-alkoxycarbonyloxy, (C1-C[lacuna])-alkylcarbonyloxy, (C1-C[lacuna])-alkylthio, (C1-C4)-alkylsulfinyl, (C1-C4)-alkylsulfonyl, (C1-C4)-alkoxy-(C1-C4)-alkyl, amino, mono-(C1-C[lacuna])-alkylamino, di-(C1-C)-alkyl)-amino, where the two C1-C4-alkyl radicals together may form a ring, which optionally contains one or more NH, N-(C1-C4)-alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkylcarbonyl, (C1-C6)-alkoxycarbonyl or hydroxyl;

A, B, C and D independently of one another are a nitrogen atom (in which case R3, R4, R5 and R6 represent the free electron pair at the nitrogen atom) or are a carbon atom substituted by one of the radicals R3-R6;

R3, R4, R5 and R6 independently of one another are, when attached to nitrogen, a free electron pair, or, when attached to carbon, hydrogen, halogen, cyano, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy (C2-C6)-alkenyl, (C2-C6)-alkynyl, (C3-C8)-cycloalkyl, straight-chain or branched (C1-C6)-alkoxy, straight-chain or branched (C1-C6)-alkylenedioxy, (C1-C6)-alkoxycarbonyloxy, (C1-C6)-alkylcarbonyloxy, (C1-C[lacuna])-alkylthio, (C1-C4)-alkylsulfinyl, (C1-C4)-alkylsulfonyl, carboxyl, (C1-C6)-alkyl carboxylate, carboxamide, N-(C1-C4)-alkyl-carboxamide, N,N-di-(C1-C4)-alkyl-carboxamide, (C1-C6)-alkoxy-(C1-C6)-alkyl, amino, mono-(C1-C6)-alkylamino, di-(C1-C6)-alkyl)-amino, where the two C1-C4-alkyl radicals together may form a ring, which optionally contains one or more NH, N-(C1-C4)-alkyl, O or S, aryl, aryloxy, aryl-(C1-C4)-alkyl, aryl-(C1-C4)-alkoxy-(C1-C4)-alkyl, (C1-C6)-alkylcarbonyl, (C1-C6)-alkoxycarbonyl, hydroxyl, where two directly adjacent radicals may be attached to one another;

Y is unsubstituted (C10-C14)-aryl or (C10-C14)-aryl which is fully or partially substituted by identical or different substituents, preferably 1- or 2-naphthyl, or is unsubstituted (C1-C13)-heteroaryl or (C1-C13)-heteroaryl which is fully or partially substituted by identical or different substituents and has in each case at least one to four N, NH, N-(C1-C6)-alkyl, O and/or S as ring members, or is unsubstituted (C3-C8)-cycloalkyl or (C3-C8)-cycloalkyl which is fully or partially substituted by identical or different substituents, where the identical or different substituents are selected independently of one another from the group consisting of halogen, preferably fluorine, chlorine, bromine or iodine; cyano; straight-chain or branched cyano-(C1-C6)-alkyl; hydroxyl; straight-chain or branched (C1-C6)-alkyl which is substituted by one or more hydroxyl substituents; carboxyl; (C1-C6)-alkyl carboxylate; carboxamide; N-(C1-C6)-alkyl-carboxamide, N,N-di-(C1-C4)-alkyl-carboxamide, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, straight-chain or branched (C2-C6)-alkenyl, straight-chain or branched (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, thio (—SH), straight-chain or branched (C1-C6)-alkylthio, (C1-C6)-alkylsulfinyl, (C1-C6)-alkylsulfonyl, (C1-C6)-alkoxy-(C1-C6)-alkyl, amino, straight-chain or branched mono-(C1-C6)-alkylamino, straight-chain or branched N,N-di-(C1-C6)-alkylamino, where the two (C1-C6)-alkyl radicals together may form a ring, which may optionally contain one or more NH, N-(C1-C6)-alkyl, O and/or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkoxycarbonyl, straight-chain or branched mono- and N,N-di-(C1-C6)-alkylcarbonylamino, straight-chain or branched mono-N- and N,N-di-(C1-C6)-alkoxycarbonylamino, straight-chain or branched N-(C1-C6)-alkylcarbonyl-N-(C1-C6)-alkylamino, straight-chain or branched N-(C1-C6)-alkoxycarbonyl-N-(C1-C6)-alkylamino, formylamino, formyl, where two directly adjacent radicals may be attached to one another;

X is an oxygen or sulfur atom, is NH, or is a generally (at the same C atom) substituted hydroxyl and hydrogen (—CH(OH)—);

their stereoisomers, their tautomers, and the pharmaceutically acceptable salts thereof, except for the compounds according to formula I where R1=R2=R3=R5=R6=hydrogen, X=oxygen or, if R4=H, generally substituted hyroxyl [sic] and hydrogen, Y=3-carboxypyridin-4-yl and R4=hydrogen or methoxy, and including the compounds 2-cyclopropylcarbonylindole and 2-cyclohexylcarbonylindole, are provided for use as a medicament, in particular an antitumor agent.

The compounds according to the invention of the formula I can be prepared by processes known per se, for example by the following processes:

a) Lithiation of the indole derivatives and conversion into the corresponding methanones:

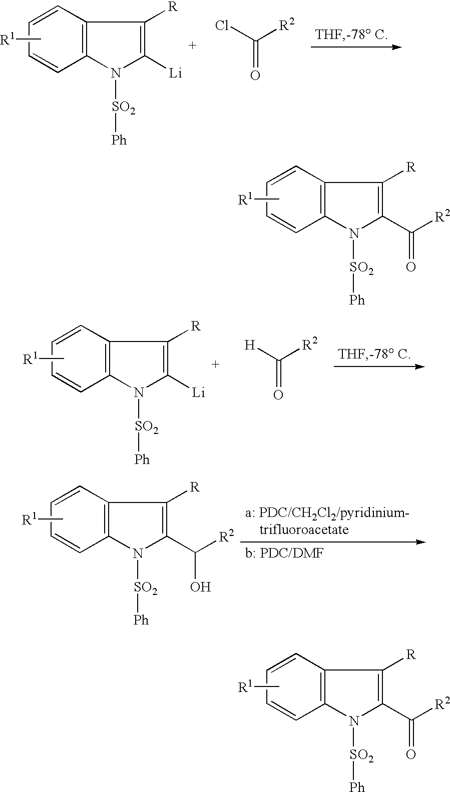

b) Removal of the phenylsulfonyl protective group:

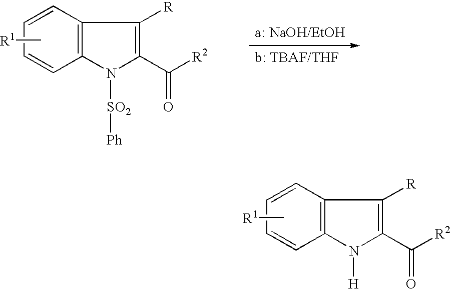

c) Further reaction of the methanones for R¹=5-benzyloxy:

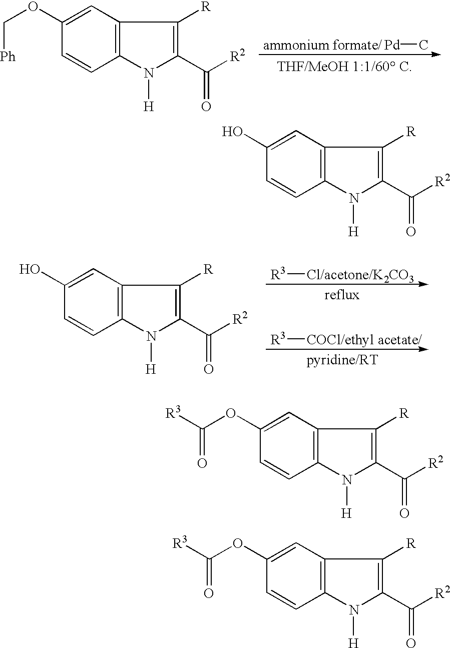

The compounds of the above formula I in which R1 is hydrogen or a phenylsulfonyl radical are useful intermediates for preparing the other compounds of the formula I.

The compounds used as starting materials, some of which are commercially available or known from the literature, are obtained by processes known from the literature; furthermore, their preparation is described in the examples. The processes known from the literature are described, for example, in L. and M. Fieser, Organische Chemie [Organic Chemistry], 2nd edition, 1979, pages 1417 to 1483 and in the literature cited therein on pages 1481-1483, in Houben-Weyl-Müller, Methoden der organischen Chemie [Methods of Organic Chemistry] and in Ullmanns Encyklopadie der technischen Chemie [Ullmann's Encyclopedia of Technical Chemistry].

Furthermore, the resulting compounds of the formula I can be separated into their enantiomers and/or diastereomers. Thus, for example, the resulting compounds of the formula I which occur as racemates can be separated into their optical antipodes by methods known per se, and compounds of the formula I having at least two asymmetrically substituted carbon atoms can be separated owing to their physico-chemical differences by methods known per se, for example by chromatography and/or fractional crystallization, into their diastereomers which, if obtained in racemic form, can then be separated into the enantiomers as mentioned above.

Separation of enantiomers is preferably carried out by column chromatography on chiral phases or by recrystallization from an optically active solvent or by reaction with an optically active substance which forms salts or derivatives, such as, for example, esters or amides, with the racemic compound.

Furthermore, the resulting compounds of the formula I can be converted into their salts with inorganic or organic acids, in particular, for pharmaceutical use, into their pharmacologically and physiologically acceptable salts. Acids which are suitable for this purpose are, for example, hydrochloric acid, hydrobromic acid, sulfuric acid, phosphoric acid, fumaric acid, succinic acid, lactic acid, citric acid, tartaric acid or maleic acid.

Moreover, if they contain an acidic group, such as a carboxyl group, the compounds of the formula I can, if desired, be converted into their salts with inorganic or organic bases, in particular, for pharmaceutical use, into their physiologically acceptable salts. Bases which are suitable for this purpose are, for example, sodium hydroxide, potassium hydroxide, cyclohexylamine, ethanolamine, diethanolamine and triethanolamine.

As mentioned at the outset, the novel compounds of the formula I and their salts have useful properties. Thus, the compounds of the formula I according to the invention have, for example, useful pharmacological properties. In particular, the compounds of the formula I can be used as antitumor agents and for the chemotherapy of tumor patients. The compounds of the formula I inhibit cell division (anti-mitosis action) and thus tumor growth. In addition, the compounds according to the invention can inhibit tubulin polymerization indirectly or directly. Inhibition of cell division may be effected by stopping the cell cycle of the tumor cells, resulting in the death of the cells (apoptosis). The compounds of the formula I are furthermore suitable for preventing or reducing formation and proliferation of metastases in the body. Moreover, they have anti-angiogenic potential and may therefore be suitable for use as antitumor agents, by inhibiting tumor vascularization.

### The examples below illustrate the invention without limiting it.

### General Procedure for Preparing the 1-Phenylsulfonyl-1H-2-indolylphenyl-1-methanols According to the Invention

At −78° C., 9.9 ml (15.9 mmol) of n-butyllithium are added dropwise to 2.23 ml (15.9 mmol) of abs. diisopropylamine in 15 ml of abs. THF. The mixture is stirred at this temperature for 10 min and then warmed to 0° C. and stirred for a further 30 min. A solution of the appropriate 1-phenylsulfonylindole (component A) (14.0 mmol) in 22 ml of abs. THF is added over a period of 10 min. The reaction mixture is stirred at 0° C. for 30 min and then cooled to −78° C. The appropriate aldehyde (component B) (15.4 mmol) is dissolved in 15 ml of abs. THF and added dropwise. After warming to room temperature (overnight), the mixture is poured into into 100 ml of 1% HCl. The organic phase is separated off and the,aqueous phase is extracted three times with in each case 50 ml of ethyl acetrate [sic]. The combined organic phases are washed with 10% sodium bicarbonate solution and water and dried over sodium sulfate. The solvent is removed under reduced pressure and the crude product is then purified by column chromatography or recrystallized from ethanol.

### EXAMPLE 1

Component A: 5-methoxy-1-phenylsulfonyl-1H-2-indole

Component B: benzaldehyde

5-methoxy-1-phenylsulfonyl-1H-2-indolylphenyl-1-methanol

Mp.: 51-52° C.

### EXAMPLE 2

Component A: 5-methoxy-1-phenylsulfonyl-1H-2-indole

Component B: 2-methoxy-benzaldehyde

5-methoxy-1-phenylsulfonyl-1H-2-indolyl(2-methoxyphenyl)-1-methanol

Mp.: 75-76° C.

### EXAMPLE 3

Component A: 5-methoxy-1-phenylsulfonyl-1H-2-indole

Component B: 3-methoxy-benzaldehyde

5-methoxy-1-phenylsulfonyl-1H-2-indolyl(3-methoxyphenyl)-1-methanol

Mp.: 121-122° C.

### EXAMPLE 4

Component A: 5-methoxy-1-phenylsulfonyl-1H-2-indole

Component B: 4-methoxy-benzaldehyde

5-methoxy-1-phenylsulfonyl-1H-2-indolyl(4-methoxyphenyl)-1-methanol

Mp.: 78-79° C.

### EXAMPLE 5

Component A: 5-methoxy-1-phenylsulfonyl-1H-2-indole

Component B: 2,4-dimethoxy-benzaldehyde

5-methoxy-1-phenylsulfonyl-1H-2-indolyl(2,4-dimethoxyphenyl)-1-methanol

Mp.: 119-120° C.

### EXAMPLE 6

Component A: 1-phenylsulfonyl-1H-2-indole

Component B: 3-pyridinyl-carbaldehyde

1-phenylsulfonyl-1H-2-indolyl(3-pyridinyl)-1-methanol

Mp.: 146° C. (decomp.)

### EXAMPLE 7

Component A: 4-hydroxy(1-phenylsulfonyl-1H-2-indole)

Component B: 4-cyanobenzaldehyde

4-hydroxy(1-phenylsulfonyl-1H-2-indolyl)methyl-1-benzenecarbonitrile

Mp.: 150° C. (decomp.)

### EXAMPLE 8

Component A: 5-methoxy-1-phenylsulfonyl-1H-2-indole

Component B: 4-isoquinolinyl-carbaldehyde

4-isoquinolinyl(5-methoxy-1-phenylsulfonyl-1H-2-indolyl)-1-methanol

Mp.: 138-139° C.

### EXAMPLE 9

Component A: 5-methoxy-1-phenylsulfonyl-1H-2-indole

Component B: 1-isoquinolinylcarbaldehyde

1-isoquinolinyl(5-methoxy-1-phenylsulfonyl-1H-2-indolyl)-1-methanol

Mp.: 167-168° C.

### General Procedure for Preparing the 1-Phenylsulfonyl-1H-2-indolylphenyl-1-methanones According to the Invention

17.8 ml (28.6 mmol) of n-butyllithium are added dropwise to 4.01 ml (28.6 mmol) of abs. diisopropylamine in 30 ml of abs. THF. The mixture is stirred at this temperature for 10 min and then warmed to 0° C. A solution of the appropriate 1-phenyl-sulfonylindole (component A) (26.0 mmol) in 35 ml of abs. THF is added over a period of 10 min. The reaction mixture is stirred at 0° C. for 60 min and then cooled to −78° C.

This mixture is added to a solution, precooled to −78° C., of the appropriate carbonyl chloride (component B) (30 mmol) in 40 ml of abs. THF. The mixture is stirred at this temperature for 60 min and then poured into 200 ml of 5% sodium bicarbonate solution and extracted with ethyl acetate. The organic phase is dried over sodium sulfate and the solvent is removed under reduced pressure. The residue is dissolved in ether and mixed with petroleum ether until crystallization sets in. The product is filtered off, washed with petroleum ether and dried.

### EXAMPLE 10

Component A: 1-phenylsulfonyl-1H-2-indole

Component B: benzoyl chloride

1-phenylsulfonyl-1H-2-indolylphenyl-1-methanone

Mp.: 142-143° C.

### EXAMPLE 11

Component A: 1-phenylsulfonyl-1H-2-indole

Component B: 2-methoxy-benzoyl chloride

1-phenylsulfonyl-1H-2-indolyl(2-methoxyphenyl)-1-methanone

Mp.: 141-143° C.

### EXAMPLE 12

Component A: 1-phenylsulfonyl-1H-2-indole

Component B: 3-methoxy-benzoyl chloride

1-phenylsulfonyl-1H-2-indolyl(3-methoxyphenyl)-1-methanone

Mp.: 101-103° C.

### EXAMPLE 13

Component A: 1-phenylsulfonyl-1H-2-indole

Component B: 2,4-dimethoxy-benzoyl chloride

1-phenylsulfonyl-1H-2-indolyl(2,4-dimethoxyphenyl)-1-methanone

Mp.: 66-68° C.

### EXAMPLE 14

Component A: 1-phenylsulfonyl-1H-2-indole

Component B: 3,4,5-trimethoxy-benzoyl chloride

1-phenylsulfonyl-1H-2-indolyl(3,4,5-trimethoxyphenyl)-1-methanone

Mp.: 152-153° C.

### EXAMPLE 15

Component A: 3-methyl-1-phenylsulfonyl-1H-2-indole

Component B: 2-methoxy-benzoyl chloride 3-methyl-1-phenylsulfonyl-1H-2-indolyl(2-methoxyphenyl)-1-methanone

Mp.: 167-169° C.

### EXAMPLE 16

Component A: 3-methyl-1-phenylsulfonyl-1H-2-indole

Component B: 3-methoxy-benzoyl chloride

3-methyl-1-phenylsulfonyl-1H-2-indolyl(3-methoxyphenyl)-1-methanone

Mp.: 113° C.

### EXAMPLE 17

Component A: 3-methyl-1-phenylsulfonyl-1H-2-indole

Component B: 2,4-dimethoxy-benzoyl chloride 3-methyl-1-phenylsulfonyl-1H-2-indolyl(2,4-dimethoxyphenyl)-1-methanone

Mp.: 155-157° C.

### EXAMPLE 18

Component A: 3-methyl-1-phenylsulfonyl-1H-2-indole

Component B: 3,4,5-trimethoxy-benzoyl chloride 3-methyl-1-phenylsulfonyl-1H-2-indolyl(3,4,5-trimethoxyphenyl)-1-methanone

### EXAMPLE 19

Component A: 5-methyl-1-phenylsulfonyl-1H-2-indole

Component B: 2-methoxy-benzoyl chloride

5-methyl-1-phenylsulfonyl-1H-2-indolyl(2-methoxyphenyl)-1-methanone

Mp.: 157-158° C.

### EXAMPLE 20

Component A: 5-methyl-1-phenylsulfonyl-1H-2-indole

Component B: 3-methoxy-benzoyl chloride

5-methyl-1-phenylsulfonyl-1H-2-indolyl(3-methoxyphenyl)-1-methanone

Mp.: 124-127° C.

### EXAMPLE 21

Component A: 5-methyl-1-phenylsulfonyl-1H-2-indole

Component B: 2,4-dimethoxy-benzoyl chloride

5-methyl-1-phenylsulfonyl-1H-2-indolyl(2,4-dimethoxyphenyl)-1-methanone

### EXAMPLE 22

Component A: 5-methyl-1-phenylsulfonyl-1H-2-indole

Component B: 3,4,5-trimethoxy-benzoyl chloride

5-methyl-1-phenylsulfonyl-1H-2-indolyl(3,4,5-trimethoxyphenyl)-1-methanone

### EXAMPLE 23

Component A: 5-methoxy-1-phenylsulfonyl-1H-2-indole

Component B: benzoyl chloride

5-methoxy-1-phenylsulfonyl-1H-2-indolylphenyl-1-methanone

Mp.: 148° C.

### EXAMPLE 24

Component A: 5-methoxy-1-phenylsulfonyl-1H-2-indole

Component B: 2-methoxy-benzoyl chloride

5-methoxy-1-phenylsulfonyl-1H-2-indolyl(2-methoxy-phenyl)-1-methanone

Mp.: 179° C.

### EXAMPLE 25

Component A: 5-methoxy-1-phenylsulfonyl-1H-2-indole

Component B: 3-methoxy-benzoyl chloride

5-methoxy-1-phenylsulfonyl-1H-2-indolyl(3-methoxy-phenyl)-1-methanone

Mp.: 181° C.

### EXAMPLE 26

Component A: 5-methoxy-1-phenylsulfonyl-1H-2-indole

Component B: 4-methoxy-benzoyl chloride

5-methoxy-1-phenylsulfonyl-1H-2-indolyl(4-methoxy-phenyl)-1-methanone

Mp.: 129-130° C.

### EXAMPLE 27

Component A: 5-methoxy-1-phenylsulfonyl-1H-2-indole

Component B: 2,4-dimethoxy-benzoyl chloride

5-methoxy-1-phenylsulfonyl-1H-2-indolyl(2,4-dimethoxy-phenyl)-1-methanone

Mp.: 62-64° C.

### EXAMPLE 27A

Component A: 5-methoxy-1-phenylsulfonyl-1H-2-indole

Component B: 3,4-dimethoxy-benzoyl chloride

5-methoxy-1-phenylsulfonyl-1H-2-indolyl(3,4-dimethoxyphenyl)-1-methanone

Mp.: 75° C. (Decomp.)

### EXAMPLE 27B

Component A: 5-methoxy-1-phenylsulfonyl-1H-2-indole

Component B: 3,5-dimethoxy-benzoyl chloride

5-methoxy-1-phenylsulfonyl-1H-2-indolyl(3,5-dimethoxyphenyl)-1-methanone

Mp.: 122-123° C.

### EXAMPLE 28

Component A: 1-phenylsulfonyl-1H-2-indole

Component B: 3-pyridinyl-carbonyl chloride

1-phenylsulfonyl-1H-2-indolyl(3-pyridinyl)-1-methanone

Mp.: 124-125° C.

### EXAMPLE 29

Component A: 5-methoxy-1-phenylsulfonyl-1H-2-indole

Component B: 2-pyridinyl-carbonyl chloride

5-methoxy-1-phenylsulfonyl-1H-2-indolyl(2-pyridinyl)-1-methanone

Mp.: 207° C.

### EXAMPLE 30

Component A: 4-(1-phenylsulfonyl-1H-2-indole

Component B: 4-cyano-benzoyl chloride

4-(1-phenylsulfonyl-1H-2-indolylcarbonyl)-1-benzol-carbonitrile

Mp.: 175-177° C.

### EXAMPLE 31

Component A: 2-fluorophenyl(5-methoxy-1-phenylsulfonyl-1H-2-indole)

Component B: 2-fluoro-benzoyl chloride

2-fluorophenyl(5-methoxy-1-phenylsulfonyl-1H-2-indolyl)-1-methanone

Mp.: 199-205° C.

### EXAMPLE 32

Component A: 5-methoxy-1-phenylsulfonyl-1H-2-indole

Component B: 2,6-difluoro-benzoyl chloride

2,6-difluorophenyl(5-methoxy-1-phenylsulfonyl-1H-2-indolyl)-1-methanone

Mp.: 124° C.

### EXAMPLE 33

Component A: 5-methoxy-1-phenylsulfonyl-1H-2-indole

Component B: 2-methyl-benzoyl chloride

5-methoxy-1-phenylsulfonyl-1H-2-indolyl(2-methylphenyl)-1-methanone

Mp.: 149-153° C.

### EXAMPLE 34

Component A: 5-methoxy-1-phenylsulfonyl-1H-2-indole

Component B: 3-trifluoromethylphenyl-benzoyl chloride

5-methoxy-1-phenylsulfonyl-1H-2-indolyl (3-trifluoromethylphenyl)-7-methanone

Mp.: 175-177° C.

### EXAMPLE 35

Component A: 4-fluorophenyl(5-methoxy-1-phenylsulfonyl-1H-2-indole

Component B: 4-fluoro-benzoyl chloride

4-fluorophenyl(5-methoxy-1-phenylsulfonyl-1H-2-indolyl)-1-methanone

Mp.: 123-128° C.

### EXAMPLE 36

Component A: 5-methoxy-1-phenylsulfonyl-1H-2-indole

Component B: 3,4-dichloro-benzoyl chloride

5-methoxy-1-phenylsulfonyl-1H-2-indolyl(3,4-dichlorophenyl)-1-methanone

Mp.: 141-144° C.

### EXAMPLE 37

Component A: 5-methoxy-1-phenylsulfonyl-1H-2-indole

Component B: 4-chloro-benzoyl chloride

5-methoxy-1-phenylsulfonyl-1H-2-indolyl(4-chlorophenyl)-1-methanone

Mp.: 146-148° C.

### EXAMPLE 38

Component A: 5-methoxy-1-phenylsulfonyl-1H-2-indole

Component B: 4-bromo-benzoyl chloride

5-methoxy-1-phenylsulfonyl-1H-2-indolyl(4-bromophenyl)-1-methanone

Mp.: 145-148° C.

### EXAMPLE 39

Component A: 5-methoxy-1-phenylsulfonyl-1H-2-indole

Component B: 3,4,5-trimethoxy-benzoyl chloride

5-methoxy-1-phenylsulfonyl-1H-2-indolyl(3,4,5-trimethoxyphenyl)-1-methanone

Mp.: 140-142° C.

### EXAMPLE 40

Component A: 5-methoxy-1-phenylsulfonyl-1H-2-indole

Component B: 4-pentyloxy-benzoyl chloride

5-methoxy-1-phenylsulfonyl-1H-2-indolyl(4-pentyloxyphenyl)-1-methanone

Mp.: 118-120° C.

### EXAMPLE 41

Component A: 5-methoxy-1-phenylsulfonyl-1H-2-indole

Component B: 1-naphthyl-carbonyl chloride

5-methoxy-1-phenylsulfonyl-1H-2-indolyl(1-naphthalenyl)-1-methanone

Mp.: 225-228° C.

### EXAMPLE 42

Component A: 5-methoxy-1-phenylsulfonyl-1H-2-indole

Component B: 4-tert-buty-benzoyl chloride

4-tert-butylphenyl(5-methoxy-1-phenylsulfonyl-1H-2-indolyl-1-methanone)

Mp.: 161-163° C.

### EXAMPLE 43

Component A: 5-methoxy-1-phenylsulfonyl-1H-2-indole

Component B: 2,3-dimethoxy-benzoyl chloride

5-methoxy-1-phenylsulfonyl-1H-2-indolyl(2,3-dimethoxyphenyl)-1-methanone

Mp.: 128° C.

### EXAMPLE 44

Component A: 5-methoxy-1-phenylsulfonyl-1H-2-indole

Component B: 2,3,4-trimethoxy-benzoyl chloride

5-methoxy-1-phenylsulfonyl-1H-2-indolyl(2,3,4-trimethoxyphenyl)-1-methanone

Mp.: 57-59° C.

### EXAMPLE 45

Component A: 5-methoxy-1-phenylsulfonyl-1H-2-indole

Component B: 4-methyl-benzoyl chloride

5-methoxy-1-phenylsulfonyl-1H-2-indolyl(4-methylphenyl)-1-methanone

Mp.: 126-127° C.

### EXAMPLE 46

Component A: 5-methoxy-1-phenylsulfonyl-1H-2-indole

Component B: 4-ethyl-benzoyl chloride

5-methoxy-1-phenylsulfonyl-1H-2-indolyl(4-ethylphenyl)-1-methanone

Mp.: 107-108° C.

### EXAMPLE 47

Component A: 5-methoxy-1-phenylsulfonyl-1H-2-indole

Component B: 4-propyl-benzoyl chloride

5-methoxy-1-phenylsulfonyl-1H-2-indolyl(4-propylphenyl)-1-methanone

Mp.: 112-114° C.

### EXAMPLE 48

Component A: 5-methoxy-1-phenylsulfonyl-1H-2-indole

Component B: 2-chloro-6-fluoro-benzoyl chloride

5-methoxy-1-phenylsulfonyl-1H-2-indolyl(2-chloro-6-fluorophenyl)-1-methanone

Mp.: 130° C.

### EXAMPLE 49

Component A: 5-methoxy-1-phenylsulfonyl-1H-2-indole

Component B: 2,5-dimethyl-benzoyl chloride

5-methoxy-1-phenylsulfonyl-1H-2-indolyl(2,5-dimethylphenyl)-1-methanone

Mp.: 164° C.

### EXAMPLE 50

Component A: 5-methoxy-1-phenylsulfonyl-1H-2-indole

Component B: 2-nitro-benzoyl chloride

5-methoxy-1-phenylsulfonyl-1H-2-indolyl(2-nitrophenyl)-1-methanone

Mp.: 190-191° C.

### EXAMPLE 51

Component A: 5-methoxy-1-phenylsulfonyl-1H-2-indole

Component B: 2-amino-benzoyl chloride

5-methoxy-1-phenylsulfonyl-1H-2-indolyl(2-aminophenyl)-1-methanone

### EXAMPLE 52

Component A: 5-methoxy-1-phenylsulfonyl-1H-2-indole

Component B: 3-nitro-benzoyl chloride

5-methoxy-1-phenylsulfonyl-1H-2-indolyl(3-nitrophenyl)-1-methanone

Mp.: 228-230° C.

### EXAMPLE 53

Component A: 5-methoxy-1-phenylsulfonyl-1H-2-indole

Component B: 3-amino-benzoyl chloride

5-methoxy-1-phenylsulfonyl-1H-2-indolyl(3-aminophenyl)-1-methanone

Mp.: 188-189° C.

### EXAMPLE 54

Component A: 5-methoxy-1-phenylsulfonyl-1H-2-indole

Component B: 4-nitro-benzoyl chloride

5-methoxy-1-phenylsulfonyl-1H-2-indolyl(4-nitrophenyl)-1-methanone

Mp.: 161-162° C.

### EXAMPLE 55

Component A: 5-methoxy-1-phenylsulfonyl-1H-2-indole

Component B: 4-amino-benzoyl chloride

5-methoxy-1-phenylsulfonyl-1H-2-indolyl(4-aminophenyl)-1-methanone

### EXAMPLE 56

Component A: 5-methoxy-1-phenylsulfonyl-1H-2-indole

Component B: 3-methoxy-2-nitro-benzoyl chloride

5-methoxy-1-phenylsulfonyl-1H-2-indolyl(3-methoxy-2-nitrophenyl)-1-methanone

Mp.: 180° C.

### EXAMPLE 57

Component A: 5-methoxy-1-phenylsulfonyl-1H-2-indole

Component B: 2-amino-3-methoxy-benzoyl chloride

5-methoxy-1-phenylsulfonyl-1H-2-indolyl (2-amino-3-methoxyphenyl)-1-methanone

### EXAMPLE 58

Component A: 5-methoxy-1-phenylsulfonyl-1H-2-indole

Component B: 2-methyl-3-nitro-benzoyl chloride

5-methoxy-1-phenylsulfonyl-1H-2-indolyl (2-methyl-3-nitrophenyl)-1-methanone

Mp.: 210-211° C.

### EXAMPLE 59

Component A: 5-methoxy-1-phenylsulfonyl-1H-2-indole

Component B: 3-amino-2-methyl-benzoyl chloride

5-methoxy-1-phenylsulfonyl-1H-2-indolyl(3-amino-2-methylphenyl)-1-methanone

Mp.: 206-207° C.

### EXAMPLE 60

Component A: 5-methoxy-1-phenylsulfonyl-1H-2-indole

Component B: cyclopropylcarbonyl chloride

cyclopropyl(5-methoxy-1-phenylsulfonyl-1H-2-indolyl)-1-methanone

Mp.: 118-120° C.

### EXAMPLE 61

Component A: 5-methoxy-1-phenylsulfonyl-1H-2-indole

Component B: cyclobutylcarbonyl chloride

cyclobutyl(5-methoxy-1-phenylsulfonyl-1H-2-indolyl)-1-methanone

Mp.: 146-147° C.

### EXAMPLE 62

Component A: 5-benzyloxy-1-phenylsulfonyl-1H-2-indole

Component B: benzoyl chloride

5-benzyloxy-1-phenylsulfonyl-1H-2-indolylphenyl-1-methanone

Mp.: 205-207° C.

### EXAMPLE 63

Component A: 5-benzyloxy-1-phenylsulfonyl-1H-2-indole

Component B: 3-chloro-benzoyl chloride

5-benzyloxy-1-phenylsulfonyl-1H-2-indolyl(3-chlorophenyl)-1-methanone

Mp.: 150-152° C.

### EXAMPLE 64

Component A: 5-benzyloxy-1-phenylsulfonyl-1H-2-indole

Component B: 4-chloro-benzoyl chloride

5-benzyloxy-1-phenylsulfonyl-1H-2-indolyl(4-chlorophenyl)-1-methanone

Mp.: 63-65° C.

### EXAMPLE 65

Component A: 5-benzyloxy-1-phenylsulfonyl-1H-2-indole

Component B: 4-methoxy-benzoyl chloride

5-benzyloxy-1-phenylsulfonyl-1H-2-indolyl(4-methoxyphenyl)-1-methanone

Mp.: 70-72° C.

### EXAMPLE 66

Component A: 5-benzyloxy-1-phenylsulfonyl-1H-2-indole

Component B: 3,4,5-trimethoxy-benzoyl chloride

5-benzyloxy-1-phenylsulfonyl-1H-2-indolyl(3,4,5-trimethoxyphenyl)-1-methanone

Mp.: 150-152° C.

### EXAMPLE 67

Component A: 5-benzyloxy-1-phenylsulfonyl-1H-2-indole

Component B: 2-methoxy-benzoyl chloride

5-benzyloxy-1-phenylsulfonyl-1H-2-indolyl-(2-methoxyphenyl)-1-methanone

Mp.: 115-116° C.

### EXAMPLE 68

Component A: 5-benzyloxy-1-phenylsulfonyl-1H-2-indole

Component B: 3-methoxy-benzoyl chloride

5-benzyloxy-1-phenylsulfonyl-1H-2-indolyl-(3-methoxyphenyl)-1-methanone

Mp.: 129-131° C.

### EXAMPLE 69

Component A: 5-methoxy-1-phenylsulfonyl-1H-2-indole

Component B: 4-isoquinolyl-carbonyl chloride

4-isoquinolyl(5-methoxy-1-phenylsulfonyl-1H-2-indolyl)-1-methanone

Mp.: 189-190° C.

### EXAMPLE 70

Component A: 5-methoxy-1-phenylsulfonyl-1H-2-indole

Component B: 1-isoquinolyl-carbonyl chloride

1-isoquinolyl(5-methoxy-1-phenylsulfonyl-1H-2-indolyl)-1-methanone

Mp.: 200° C.

### EXAMPLE 71

Component A: 1-phenylsulfonyl-1H-pyrrolo(2,3-b]pyridine

Component B: 2-methoxy-benzoyl chloride

1-phenylsulfonyl-1H-pyrrolo[2,3-b]pyridin-2-yl(2-methoxyphenyl)-1-methanone

Mp.: 124-125° C.

### EXAMPLE 72

Component A: 1-phenylsulfonyl-1H-pyrrolo[2,3-b]pyridine

Component B: 3-methoxy-benzoyl chloride

1-phenylsulfonyl-1H-pyrrolo[2,3-b]pyridin-2-yl(3-methoxyphenyl)-1-methanone

Mp.: 139-140° C.

### EXAMPLE 73

Component A: 1-phenylsulfonyl-1H-pyrrolo[2,3-b]pyridine

Component B: 3,4,5-trimethoxy-benzoyl chloride

1-phenylsulfonyl-1H-pyrrolo[2,3-b]pyridin-2-yl(3,4,5-trimethoxyphenyl)-1-methanone

Mp.: 180-181° C.

### EXAMPLE 74

Component A: 1-phenylsulfonyl-1H-pyrrolo[2,3-b]pyridine

Component B: 2,4-dimethoxy-benzoyl chloride

1-phenylsulfonyl-1H-pyrrolo[2,3-b]pyridin-2-yl(2,4-dimethoxyphenyl)-1-methanone

Mp.: 190-195° C. (decomp.) ° C. [sic]

### EXAMPLE 75

Component A: 5-methoxy-1-phenylsulfonyl-1H-pyrrolo[2,3-b]pyridine

Component B: 2-methoxy-benzoyl chloride

5-methoxy-1-phenylsulfonyl-1H-pyrrolo[2,3-b]pyridin-2-yl(2-methoxyphenyl)-1-methanone

### EXAMPLE 76

Component A: 5-methoxy-1-phenylsulfonyl-1H-pyrrolo[2,3-b]pyridine

Component B: 3-methoxy-benzoyl chloride

5-methoxy-1-phenylsulfonyl-1H-pyrrolo[2,3-b]pyridin-2-yl(3-methoxyphenyl)-1-methanone

### EXAMPLE 77

Component A: 5-methoxy-1-phenylsulfonyl-1H-pyrrolo[2,3-b]pyridine

Component B: 3,4,5-trimethoxy-benzoyl chloride

5-methoxy-1-phenylsulfonyl-1H-pyrrolo[2,3-b]pyridin-2-yl(3,4,5-trimethoxyphenyl)-1-methanone

### EXAMPLE 78

Component A: 5-methoxy-1-phenylsulfonyl-1H-pyrrolo[2,3-b]pyridine

Component B: 2,4-dimethoxy-benzoyl chloride

5-methoxy-1-phenylsulfonyl-1H-pyrrolo[2,3-b]pyridin-2-yl(2,4-dimethoxyphenyl)-1-methanone

### EXAMPLE 79

Component A: 5-methoxy-1-phenylsulfonyl-1H-pyrrolo[3,2-b]pyridine

Component B: benzoyl chloride

5-methoxy-1-phenylsulfonyl-1H-pyrrolo[3,2-b]pyridin-2-ylphenyl-1-methanone

### EXAMPLE 80

Component A: 5-methoxy-1-phenylsulfonyl-1H-pyrrolo[3,2-b]pyridine

Component B: 2-methoxy-benzoyl chloride

5-methoxy-1-phenylsulfonyl-1H-pyrrolo[3,2-b]pyridin-2-yl(2-methoxyphenyl)-1-methanone

### EXAMPLE 81

Component A: 5-methoxy-1-phenylsulfonyl-1H-pyrrolo[2,3-c]pyridine

Component B: 3-methoxy-benzoyl chloride

5-methoxy-1-phenylsulfonyl-1H-pyrrolo[2, 3-c]pyridin-2-yl(3-methoxyphenyl)-1-methanone

### EXAMPLE 82

Component A: 5-methoxy-1-phenylsulfonyl-1H-pyrrolo[2,3-c]pyridine

Component B: 2,4-dimethoxy-benzoyl chloride

5-methoxy-1-phenylsulfonyl-1H-pyrrolo[2, 3-c]pyridin-2-yl(2, f4-dimethoxyphenyl)-1-methanone

### EXAMPLE 83

Component A: 5-methoxy-1-phenylsulfonyl-1H-pyrrolo[2,3-c]pyridine

Component B: 3,4,5-trimethoxy-benzoyl chloride

5-methoxy-1-phenylsulfonyl-1H-pyrrolo[2,3-c]pyridin-2-yl(3,4,5-trimethoxyphenyl)-1-methanone

### EXAMPLE 84

Component A: 5-methoxy-1-phenylsulfonyl-1H-pyrrolo[2,3-b]pyridine

Component B: 2-methoxy-benzoyl chloride

5-methoxy-1-phenylsulfonyl-1H-pyrrolo[3,2-b]pyridin-2-yl(2-methoxyphenyl-1-methanones [sic]

Mp.: 197-198° C.

### EXAMPLE 85

Component A: 5-methoxy-1-phenylsulfonyl-1H-pyrrolo(3,2-b]pyridine

Component B: 3-methoxy-benzoyl chloride

5-methoxy-1-phenylsulfonyl-1H-pyrrolo[3,2-b]pyridin-2-yl(3-methoxyphenyl-1-methanones [sic]

Mp.: 147-149° C.

### EXAMPLE 86

Component A: 5-methoxy-1-phenylsulfonyl-1H-pyrrolo[3,2-b]pyridine

Component B: 2,4-dimethoxy-benzoyl chloride

5-methoxy-1-phenylsulfonyl-1H-pyrrolo[3,2-b)pyridin-2-yl(2,4-dimethoxyphenyl-1-methanones [sic]

Mp.: 132° C.

### EXAMPLE 87

Component A: 5-methoxy-1-phenylsulfonyl-1H-pyrrolo3,2-b]pyridine

Component B: 3,4,5-trimethoxy-benzoyl chloride

5-methoxy-1-phenylsulfonyl-1H-pyrrolo[3,2-b]pyridin-2-yl(3,4,5-trimethoxyphenyl-1-methanones [sic]

Mp.: 190-191° C.

### General Procedures for Preparing the 1H-2-Indolylphenyl-1-methanones According to the Invention

Method A: The appropriate N-protected methanone derivative (starting component) (1.8 mmol) is, in a mixture of 10% sodium hydroxide (20 ml) and ethanol (40 ml), heated at reflux for 2 to 15 hours (TLC). The solution is cooled to room temperature and then poured into 100 ml of water and extracted with ethyl acetate. The organic phase is dried over sodium sulfate and the solvent is removed. The crude product is recrystallized from ethyl acetate.

Method B: A mixture of the appropriate N-protected methanone derivative (starting component) (1.8 mmol) and 0.79 g (2.5 mmol) of tetrabutylammonium fluoride trihydrate in 20 ml of THF/methanol 1:1 is heated at reflux. After the reaction has ended (30 min-4 hours, TLC), the mixture is cooled and poured into 100 ml of water. The mixture is extracted with ethyl acetate and the organic phase is dried over sodium sulfate. The solvent is concentrated slowly until the product begins to crystallize out.

### EXAMPLE 88

Starting component: compound according to Example 10

Method A or B

1H-2-indolylphenyl-1-methanone

Mp.: 145-147° C.

### EXAMPLE 89

Starting component: compound according to Example 11

Method A or B

1H-2-indolyl(2-methoxyphenyl)-1-methanone

Mp.: 129-130° C.

### EXAMPLE 90

Starting component: compound according to Example 12

Method A or B

1H-2-indolyl(3-methoxyphenyl)-1-methanone

Mp.: 124-126° C.

### EXAMPLE 91

Starting component: compound according to Example 13

Method A or B

1H-2-indolyl(2,4-dimethoxyphenyl)-1-methanone

Mp.: 134-135° C.

### EXAMPLE 92

Starting component: compound according to Example 14

Method A or B

1H-2-indolyl(3,4,5-trimethoxyphenyl)-1-methanone

Mp.: 148-150° C.

### EXAMPLE 93

Starting component: compound according to Example 15

Method A or B

3-methyl-1H-2-indolyl(2-methoxyphenyl)-1-methanone

Mp.: 152-153° C.

### EXAMPLE 94

Starting component: compound according to Example 16

Method A or B

3-methyl-1H-2-indolyl(3-methoxyphenyl)-1-methanone

Mp.: 131° C.

### EXAMPLE 95

Starting component: compound according to Example 17

Method A or B

3-methyl-1H-2-indolyl(2,4-dimethoxyphenyl)-1-methanone

Mp.: 124-126° C.

### EXAMPLE 96

Starting component: compound according to Example 18

Method A or B

3-methyl-1H-2-indolyl(3,4,5-trimethoxyphenyl)-1-methanone

Mp.: 138-144° C.

### EXAMPLE 97

Starting component: compound according to Example 19

Method A or B

5-methyl-1H-2-indolyl(2-methoxyphenyl)-1-methanone

Mp.: 165-167° C.

### EXAMPLE 98

Starting component: compound according to Example 20

Method A or B

5-methyl-1H-2-indolyl(3-methoxyphenyl)-1-methanone

Mp.: 192-202° C.

### EXAMPLE 99

Starting component: compound according to Example 21

Method A or B

5-methyl-1H-2-indolyl(2,4-dimethoxyphenyl)-1-methanone

### EXAMPLE 99A

Starting component: compound according to Example XX [sic]

Method A or B

5-methyl-1H-2-indolyl(3,4-dimethoxyphenyl)-1-methanone

Mp.: 187° C.

### EXAMPLE 99B

Starting component: compound according to Example YY [sic]

Method A or B

5-methyl-1H-2-indolyl(3,5-dimethoxyphenyl)-1-methanone

Mp.: 141-142° C.

### EXAMPLE 100

Starting component: compound according to Example 22

Method A or B

5-methyl-1H-2-indolyl(3,4,5-trimethoxyphenyl)-1-methanone

Mp.: 202-203° C.

### EXAMPLE 101

Starting component: compound according to Example 23

Method A or B

5-methoxy-1H-2-indolylphenyl-1-methanone

Mp.: 162° C.

### EXAMPLE 102

Starting component: compound according to Example 24

Method A or B

5-methoxy-1H-2-indolyl(2-methoxyphenyl)-1-methanone

Mp.: 127° C.

### EXAMPLE 103

Starting component: compound according to Example 25

Method A or B

5-methoxy-1H-2-indolyl(3-methoxyphenyl)-1-methanone

Mp.: 147-148° C.

### EXAMPLE 104

Starting component: compound according to Example 26

Method A or B

5-methoxy-1H-2-indolyl(4-methoxyphenyl)-1-methanone

Mp.: 165° C.

### EXAMPLE 105

Starting component: compound according to Example 27

Method A or B

5-methoxy-1H-2-indolyl(2, 4-dimethoxyphenyl)-1-methanone

Mp.: 160-161° C.

### EXAMPLE 106

Starting component: compound according to Example 29

Method A or B

5-methoxy-1H-2-indolyl(2-pyridinyl)-1-methanone

Mp.: 201° C.

### EXAMPLE 107

Starting component: compound according to Example 30 [sic](?)

Method A or B

4-(1H-2-indolylcarbonyl)-1-benzenecarboxylic acid

Mp.:>220° C.

### EXAMPLE 108

Starting component: compound according to Example 31

Method A or B

2-fluorophenyl(5-methoxy-1H-2-indolyl)-1-methanone

Mp.: 145° C.

### EXAMPLE 109

Starting component: compound according to Example (?) [sic]

Method A or B

5-methoxy-1-phenylsulfonyl-1H-2-indolyl(3-trifluoromethylphenyl)-1-methanone

Mp.: 165° C.

### EXAMPLE 110

Starting component: compound according to Example 33

Method A or B

5-methoxy-1H-2-indolyl(2-methylphenyl)-1-methanone

Mp.: 120° C.

### EXAMPLE 111

Starting component: compound according to Example 34

Method A or B 5-methoxy-1H-2-indolyl(3-trifluoromethylphenyl)-1-methanone

Mp.: 193-195° C.

### EXAMPLE 112

Starting component: compound according to Example 35

Method A or B

4-fluorophenyl(5-methoxy-1H-2-indolyl)-1-methanone

Mp.: 168° C.

### EXAMPLE 113

Starting component: compound according to Example 36

Method A or B

5-methoxy-1H-2-indolyl(3,4-dichlorophenyl)-1-methanone

Mp.: 190-192° C.

### EXAMPLE 114

Starting component: compound according to Example 37

Method A or B

5-methoxy-1H-2-indolyl(4-chlorophenyl)-1-methanone

Mp.: 191-193° C.

### EXAMPLE 115

Starting component: compound according to Example 38

Method A or B

5-methoxy-1H-2-indolyl(4-bromophenyl)-1-methanone

Mp.: 188-190° C.

### EXAMPLE 116

Starting component: compound according to Example 39

Method A or B

5-methoxy-1H-2-indolyl(3,4,5-trimethoxyphenyl)-1-methanone

Mp.: 210-211° C.

### EXAMPLE 117

Starting component: compound according to Example 40

Method A or B

5-methoxy-1H-2-indolyl(4-pentyloxyphenyl)-1-methanone

Mp.: 139-141° C.

### EXAMPLE 118

Starting component: compound according to Example 41

Method A or B

5-methoxy-1H-2-indolyl(1-naphthalenyl)-1-methanone

Mp.: 174-175° C.

### EXAMPLE 119

Starting component: compound according to Example 42

Method A or B

4-tert-butylphenyl(5-methoxy-1H-2-indolyl-1-methanone)

Mp.: 204-207° C.

### EXAMPLE 120

Starting component: compound according to Example 43

Method A or B

5-methoxy-1H-2-indolyl(2,3-dimethoxyphenyl)-1-methanone

### EXAMPLE 121

Starting component: compound according to Example 44

Method A or B

5-methoxy-1H-2-indolyl(2,3,4-trimethoxyphenyl)-1-methanone

Mp.: 156° C.

### EXAMPLE 122

Starting component: compound according to Example 45

Method A or B

5-methoxy-1H-2-indolyl(1-methylphenyl)-1-methanone

Mp.: 200° C.

### EXAMPLE 123

Starting component: compound according to Example 46

Method A or B

5-methoxy-1H-2-indolyl(4-ethylphenyl)-1-methanone

Mp.: 154-155° C.

### EXAMPLE 124

Starting component: compound according to Example 47

Method A or B

5-methoxy-1H-2-indolyl(4-propylphenyl)-1-methanone

Mp.: 145-146° C.

### EXAMPLE 125

Starting component: compound according to Example 48

Method A or B

5-methoxy-1H-2-indolyl(2-chloro-6-fluorophenyl)-1-methanone

Mp.: 168-170° C.

### EXAMPLE 126

Starting component: compound according to Example 49

Method A or B

5-methoxy-1H-2-indolyl(2,5-dimethylphenyl)-1-methanone

Mp.: 152-153° C.

### EXAMPLE 127

Starting component: compound according to Example 50

Method A or B

5-methoxy-1H-2-indolyl(2-nitrophenyl)-1-methanone

Mp.: 185-187° C.

### EXAMPLE 128

Starting component: compound according to Example 51

Method A or B

5-methoxy-1H-2-indolyl(2-aminophenyl)-1-methanone

Mp.: 144-145° C.

### EXAMPLE 129

Starting component: compound according to Example 52

Method A or B

5-methoxy-1H-2-indolyl(3-nitrophenyl)-1-methanone

Mp.: 221-222° C.

### EXAMPLE 130

Starting component: compound according to Example 53

Method A or B

5-methoxy-1H-2-indolyl(3-aminophenyl)-1-methanone

### EXAMPLE 131

Starting component: compound according to Example 54

Method A or B

5-methoxy-1H-2-indolyl(4-nitrophenyl)-1-methanone

### EXAMPLE 132

Starting component: compound according to Example 55

Method A or B

5-methoxy-1H-2-indolyl(4-aminophenyl)-1-methanone

### EXAMPLE 133

Starting component: compound according to Example 56

Method A or B

5-methoxy-1H-2-indolyl(3-methoxy-2-nitrophenyl)-1-methanone

Mp.: 212° C. (decomp.)

### EXAMPLE 134

Starting component: compound according to Example 57

Method A or B

5-methoxy-1H-2-indolyl(2-amino-3-methoxyphenyl)-1-methanone

### EXAMPLE 135

Starting component: compound according to Example 58

Method A or B

5-methoxy-1H-2-indolyl(2-methyl-3-nitrophenyl)-1-methanone

Mp.: 199-200° C.

### EXAMPLE 136

Starting component: compound according to Example 59

Method A or B

5-methoxy-1H-2-indolyl(3-amino-2-methylphenyl)-1-methanone

Mp.: 163-165° C.

### EXAMPLE 137

Starting component: compound according to Example 60

Method A or B

cyclopropyl(5-methoxy-1H-2-indolyl)-1-methanone

Mp.: 205-207° C.

### EXAMPLE 138

Starting component: compound according to Example 61

Method A or B

cyclobutyl(5-methoxy-1H-2-indolyl)-1-methanone

Mp.: 175-179° C.

### EXAMPLE 139

Starting component: compound according to Example 62

Method A or B

5-benzyloxy-1H-2-indolylphenyl-1-methanone

Mp.: 187-188° C.

### EXAMPLE 140

Starting component: compound according to Example 63

Method A or B

5-benzyloxy-1H-2-indolyl(3-chlorophenyl)-1-methanone

Mp.: 163-165° C.

### EXAMPLE 141

Starting component: compound according to Example 64

Method A or B

5-benzyloxy-1H-2-indolyl(4-chlorophenyl)-1-methanone

Mp.: 188-190° C.

### EXAMPLE 142

Starting component: compound according to Example 65

Method A or B

5-benzyloxy-1H-2-indolyl(4-methoxyphenyl)-1-methanone

Mp.: 155-157° C.

### EXAMPLE 143

Starting component: compound according to Example 66

Method A or B

5-benzyloxy-1H-2-indolyl(3,4,5-trimethoxyphenyl)-1-methanone

Mp.: 165-167° C.

### EXAMPLE 144

Starting component: compound according to Example 67

Method A or B

5-benzyloxy-1H-2-indolyl-(2-methoxyphenyl)-1-methanone

Mp.: 150-151° C.

### EXAMPLE 145

Starting component: compound according to Example 68

Method A or B

5-benzyloxy-1H-2-indolyl-(3-methoxyphenyl)-1-methanone

Mp.: 153-154° C.

### EXAMPLE 146

Starting component: compound according to Example 69

Method A or B

4-isoquinolinyl(5-methoxy-1H-2-indolyl)-1-methanone

Mp.: 228-230° C.

### EXAMPLE 147

Starting component: compound according to Example 70

Method A or B

1-isoquinolinyl(5-methoxy-1H-2-indolyl)-1-methanone

Mp.: 175° C.

### EXAMPLE 148

Starting component: compound according to Example 71

Method A or B

1H-pyrrolo[2,3-b]pyridin-2-yl(2-methoxyphenyl)-1-methanone

Mp.: 211-213° C.

### EXAMPLE 149

Starting component: compound according to Example 72

Method A or B

1H-pyrrolo[2,3-b]pyridin-2-yl(3-methoxyphenyl)-1-methanone

Mp.: 166-168° C.

### EXAMPLE 150

Starting component: compound according to Example 73

Method A or B

1H-pyrrolo[2,3-b]pyridin-2-yl(3,4,5-trimethoxyphenyl)-1-methanone

Mp.: 205-206° C.[lacuna]

### EXAMPLE 151

Starting component: compound according to Example 74

Method A or B

1H-pyrrolo[2,3-b]pyridin-2-yl(2,4-dimethoxyphenyl)-1-methanone

Mp.: 208-210° C. (decomp.)

### EXAMPLE 152

Starting component: compound according to Example 75

Method A or B

5-methoxy-1H-pyrrolo[2,3-b]pyridin-2-yl(2-methoxyphenyl)-1-methanone

### EXAMPLE 153

Starting component: compound according to Example 76

Method A or B

5-methoxy-1H-pyrrolo[2,3-b]pyridin-2-yl(3-methoxyphenyl)-1-methanone

### EXAMPLE 154

Starting component: compound according to Example 77

Method A or B

5-methoxy-1H-pyrrolo[2,3]-b)pyridin-2-yl(3,4,5-trimethoxyphenyl)-1-methanone

### EXAMPLE 155

Starting component: compound according to Example 78

Method A or B

5-methoxy-1H-pyrrolo[2,3]-b)pyridin-2-yl(2,4-dimethoxyphenyl)-1-methanone

### EXAMPLE 156

Starting component: compound according to Example 79

Method A or B

5-methoxy-1H-pyrrolo[3,2]-b)pyridin-2-ylphenyl-1-methanone

### EXAMPLE 157

Starting component: compound according to Example 80

Method A or B

5-methoxy-1H-pyrrolo[3,2-b]pyridin-2-yl(2-methoxyphenyl)-1-methanone

### EXAMPLE 158

Starting component: compound according to Example 81

Method A or B

5-methoxy-1H-pyrrolo[2,3]-pyridin-2-yl(3-methoxyphenyl)-1-methanone

### EXAMPLE 159

Starting component: compound according to Example 82

Method A or B

5-methoxy-1H-pyrrolo[2,3-c]pyridin-2-yl(2.4-dimethoxyphenyl)-1-methanone [sic]

### EXAMPLE 160

Starting component: compound according to Example 83

Method A or B

5-methoxy-1H-pyrrolo[2,3-c]pyridin-2-yl(3,4,5-trimethoxyphenyl)-1-methanone

### EXAMPLE 161

Starting component: compound according to Example 84

Method A or B

5-methoxy-1H-pyrrolo[3,2-b]pyridin-2-yl(2-methoxyphenyl-1-methanone

Mp.: 190° C.

### EXAMPLE 162

Starting component: compound according to Example 85

Method A or B

5-methoxy-1H-pyrrolo[3,2-b]pyridin-2-yl (3-methoxyphenyl-1-methanone

Mp.: 150° C.

### EXAMPLE 163

Starting component: compound according to Example 86

Method A or B

5-methoxy-1H-pyrrolo[3,2-b]pyridin-2-yl(2,4-dimethoxyphenyl-1-methanone

Mp.: 100° C. (decomp.)

### EXAMPLE 164

Starting component: compound according to Example 87

Method A or B

5-methoxy-1H-pyrrolo[3,2-b]pyridin-2-yl(3,4,5-trimethoxyphenyl-1-methanone

Mp.: 233° C.

Alternatively, the compounds according to the invention can also be prepared by reacting an N-protected substituted indole derivative with an appropriate nitrile compound according to the exemplary procedure below.

### EXAMPLE 147

### Prepared by an Alternative Process

Compound: 1-Isoquinolinyl(5-methoxy-1H-2-indolyl)-1-methanone

n-Butyllithium (5.5 mmol, 1.6 M in hexane, from Aldrich) was added dropwise to a solution, cooled to −78° C., of 1-(tert-butyloxycarbonyl)-5-methoxyindole (5 mmol) in 10 ml of dry THF. After 30 minutes at −78° C., a solution of 1-cyanoisoquinoline (7.5 mmol) dissolved in 2 ml of THF, was slowly added dropwise. The mixture was allowed to warm slowly to room temperature overnight (16 hours). The dark-brown solution was admixed with 50 ml of a mixture of trifluoroacetic acid:dichloromethane=4:1, stirred at room temperature for 90 minutes and extracted with 30 ml of dichloromethane, the organic phase was washed with water, saturated potassium carbonate solution and again water (20 ml each) and the solvent was removed under reduced pressure. The resulting brown, oil was suspended in 10 ml of ethanol and poured into 300 ml of ice-water. The green-brown precipitate was isolated by filtration and purified by column chromatography under atmospheric pressure on silica gel 60 (mobile phase diethyl ether:hexane=1:1).

Yield: 160 mg (10%) yellow needles

### General Procedure for Preparing N-Oxides of the Azaindoles and Their Derivatization

Preparation of the N-oxides:

At 0° C., 1.00 mmol of the pyridine derivative in 20 ml of dichloromethane are admixed with 2 mmol of metachloroperbenzoic acid. The mixture is allowed to warm to r.t. and stirred at this temperature for 24 h. 10 ml of conc. NaHCO₃ sltn are added, the organic phase is separated off and the aqueous phase is extracted 10 times with 25 ml of dichloromethane each. The combined org. phases are dried over MgSO₄ and the solvent is removed. The residue that remains is admixed with a little diethyl ether, giving the product as a powdery precipitate (yld: 65%).

### EXAMPLE 164 [sic]

Starting component: compound according to Example 150

1H-pyrrolo[2,3-b]pyridin-2-yl(3,4,5-trimethoxyphenyl)-1-methanone N-oxide

Mp.: 90-92° C.

Reaction of the N-oxides with acetic anhydride: 0.5 mmol of the N-oxide are mixed with 15 ml of acetic anhydride. A drop of water is added, and the mixture is then refluxed for 12 h. Once all of the starting material has reacted according to TLC, the solvent is removed under reduced pressure and the residue is taken up in a little dichloromethane and washed with NaHCO₃ solution.

The solvent is removed and the residue is admixed with diethyl ether, giving the product as a powdery precipitate (60%)

### EXAMPLE 165 [sic]

Starting component: compound according to Example XXX [sic]

6-[2-(3,4,5-trimethoxybenzoyl)-1-acetyl-1H-pyrrolo[2,3b]pyridine]ethanoate

Mp.: 151-152° C.

### General Procedure for Preparing the N-Substituted 1H-2-Indolylphenyl-1-methanones According to the Invention

A mixture of the appropriate 1H-2-indolylphenyl-1-methanone (starting material) (5.0 mol), the hydro-chloride of the appropriate aminoalkyl chloride (15.0 mmol) and 40.0 mmol of potassium carbonate in 50 ml of abs. acetone is heated at reflux for 14 hours. After cooling, the reaction mixture is poured into 250 ml of water and extracted with dichloromethane. The organic phase is dried over sodium sulfate. The solvent is removed and the residue is then purified by column chromatography.

### EXAMPLE 166 [sic]

Starting material according to Example 101

5-methoxy-1-(2-dimethylaminoethyl)-1H-2-indolylphenyl-1-methanone

Mp.: 38-40° C.

### EXAMPLE 167 [sic]

Starting material according to Example 101

5-methoxy-1-(3-dimethylaminopropyl)-1H-2-indolylphenyl-1-methanone

Mp.: 51-52° C.

### EXAMPLE 168 [sic]

Starting material according to Example 101

5-methoxy-1-(2-pyrrolidinoethyl)-1H-2-indolylphenyl1-methanone

Mp.: 68-71° C.

### EXAMPLE 169 [sic]

Starting material according to Example 101

5-methoxy-1-(2-piperidinoethyl)-1H-2-indolylphenyl-1-methanone

Mp.: 55-57° C.

### EXAMPLE 170 [sic]

Starting material according to Example 101

5-methoxy-1-(2-morpholinoethyl)-1H-2-indolylphenyl-1-methanone

Mp.: 66-68° C.

### EXAMPLE 171 [sic]

Starting material according to Example 101

5-methoxy-1-(2-phenylmethyloxyethyl)-1H-2-indolylphenyl-1-methanone

Mp.: 95-97° C.

### Results of the Pharmacological Tests

The in vitro test in selected tumor models revealed the pharmacological activities shown below.

### EXAMPLE 1

### Antitumor Action

The substances D-64131 (Ex. 101), D-68143 (Ex. 102), D-68144 (Ex. 103), D-68150 (Ex. 116) and D-68172 (Ex. 105) were tested for antiproliferative activity in a proliferation test on established tumor cell lines. In the test used, the cellular dehydrogenase activity is determined as a measure of cell vitality and, indirectly, cell numbers. The cell lines used were the human glioma cell lines A-172 (ATCC CRL-1620), U118 (ATCC HTB-15) and U373 (ATCC HTB-17), the rat glioma cell line C6 (ATCC CCL107) and the human cervical carcinoma cell line KB/HeLa (ATCC CCL17). These were very well characterized established cell lines which were obtained from ATCC and cultured.

The results summarized in Tab. 1 and FIG. 1 show a highly potent antitumor action of the substances mentioned. It has to be emphasized that the action is concentration-dependent, resulting in comparable maximum inhibitions. It was possible to determine defined activities: D-68144>D-68150≧D-64131+D-68113>D-68172 (increasing antitumor potency from D-68172 to D-68144). This order in the activity was observed in all cell lines examined and is to be judged as an indication for a defined molecular mechanism of action.

Table 1.

Antitumor potency of various derivatives in the XTT cytotoxicity test with the glioma cell lines C6, A-172, U118, U373 and the cervical carcinoma cell line HeLa/KB. What is stated is the IC₅₀ from concentration/activity experiments in nM. If the experiments were carried out more than once, the number of independent experiments is given in brackets.

| Exam- | | | | | | KB/ |
|---|---|---|---|---|---|---|
| ple | Code No. | C6 | A-172 | U118 | U373 | HeLa |
| 101 | D-64131 | 96.5 (2) | 51 | 24 | 22 | 24 (2) |
| 102 | D-68143 | 98 (2) | 73 | 28 | 29 | 35 (2) |
| 103 | D-68144 | 9.6 (2) | 15 | 8.3 | 5.0 | 6.6 |
| 116 | D-68150 | 77 | 18.5 (2) | 19.4 | 19.7 | 32 |
| 105 | D-68172 | 180 | 330 (2) | 119 (2) | 75 (2) | 107 (2) |

FIG. 1

Graphic representation of the concentration-dependent antitumor activity of different derivatives in the XTT cytotoxicity test with the KB/HeLa cervical carcinoma cell line.

[image]

[image]

### EXAMPLE 2

### Cell Cycle Analysis Using Fluorescence-Activated Cell Sorting

The substances D-64131 (Ex. 101), D-68144 (Ex. 103) and D-68150 (Ex. 116) were examined further by fluorescence-activated cell sorting (FACS) using the human glioblastoma cell line U373. The chosen method allowed the detection of a cell-cycle-specific action of the substance. To this end, the proportion of cells in phases G1, S, G2 and M of the cell cycle was determined by measuring the DNA content. The result of this analysis is summarized in FIG. 2. What is shown is the proportion of cells in the metaphase of mitotic division (M-phase of the cell cycle; 2N chromosomes). For all of the substances tested, a concentration-dependent arrest of the cells in mitosis, which correlates with the antiproliferative action shown in Table 1 and FIG. 1, is clearly detectable. Thus, the substances arrest growth by inhibiting cell division, which subsequently results in the death of the tumor cells (apoptosis).

FIG. 2

Cell cycle analysis of substance-treated U373 glioma cells by FACS What is shown is the percentage of cells having 2N chromosomes, i.e. cells in the metaphase of mitotic cell division, as a function of substance concentration.

[image]

[image]

### Comparison of the Biological Activity of the Compound D-68144 (Example 103) According to the Invention With the Compounds 1d/4d According to the Publication by Medarde et al.

The publication by M. Medarde et al. 1998, Eur. J. Med. Chem. Vol. 33, pp. 71-77 describes combretastatin analogs which have antitumor action in a proliferation assay with the tumor cell lines P388 (leukemia, murine), A549 (pulmonary carcinoma, human), HT29 (colon carcinoma, human) and Me128 (melanoma, human). The test system used is comparable to the test system described above. The tumor cells mentioned are treated with the substances for 72 h, and the cell count is determined directly (P388) or indirectly via staining with Crystal Violet (Me128, A549, HT29). In this test, the known compound 1-methyl-2-(3,4,3-trimethoxyphenyl)carbonyl-methylindole (compound 4d) shows an inhibitory activity of IC₅₀=0.3 to 0.6 μM and the known compound 1-methyl-3-(3-hydroxy-4-methoxyphenyl)carbonylmethylindole (compound 1d) shows an inhibitory activity of IC₅₀=3.6 to 8.9 μM. In contrast, the compound D-68144 according to the invention shows an inhibitory activity in various glioma lines of IC₅₀=0.005 to 0.015 μM. Surprisingly, the compound D-68144 according to the invention is, by a factor of 40-60, more active than the compound 4d described in the publication of Medarde et al.

### Description of the Methods Used

XTT Test for Cellular Dehydroqenase Activity

The adherently growing tumor cell lines C6, A-172, U118, U373 and HeLa/KB were cultivated under standard conditions in an incubator with gas inlet at 37° C., 5% CO₂ and 95% atmospheric humidity. On Test Day 1, the cells are detached using trypsine/EDTA and pelleted by centrifugation. The cell pellet is then resuspended in the respective culture medium at the appropriate cell count and transferred to a 96-well microtiter plate. The plates are then cultivated overnight in the incubator with gas inlet. The test substances are made up as 10 mM stock solutions in DMSO and, on Test Day 2, diluted with culture medium to the desired concentrations. The substances in the culture medium are then added to the cells and incubated in the incubator with gas inlet for 45 h. Cells which have not been treated with test substance serve as control. For the XTT assay, 1 mg/ml of XTT (sodium 3′-[1-(phenylaminocarbonyl)-3,4-tetrazolium]-bis(4-methoxy-6-nitro)benzenesulfonic acid) is dissolved in RPMI-1640 medium without Phenol Red. Additionally, a 0.383 mg/ml solution of PMS (N-methyldibenzopyrazine methyl sulfate) in phosphate-buffered saline (PBS) is prepared. On Test Day 4, 75 μl/well of the XTT-PMS mixture are pipetted onto the cell plates, which by now have been incubated with the test substances for 45 h. To this end, the XTT solution is mixed with the PMS solution in a ratio of 50:1 (v/v) shortly before use. The cell plates are then incubated in the incubator with gas inlet for a further 3 h, and the optical density (OD_{490 nm}) is determined in a photometer.

Using the OD_{490 nm} obtained, the inhibition in percent relative to the control is calculated and plotted semilogarithmically in the form of a concentration-activity curve. The IC₅₀ is calculated from the concentration-activity curve by regression analysis using the program Graphpad.

Cell Cycle Analysis by FACS

U373 glioma cells in adherent subconfluent culture are treated with substance for 24 h and then detached and washed 1× with PBS. A total of 5×10⁶ cells/data point are fixed in 1 ml of 80% methanol (−20° C.), kept on ice for 30 min and stored at 4° C. For FACS analysis, the cells are incubated in PBS with 0.1% of saponin, 20 μg/ml of propidium iodide and 1 mg/ml of RNAse A at 37° C. for 30 min. The cells are washed in PBS/saponin buffer and then analyzed in a Calibur flow cytometer (Becton Dickinson).

In vitro tests in selected tumor models revealed the pharmacological activities shown below.

### EXAMPLE 3

### Tubulin-Inhibitory and Cytotoxic Activity of Selected Compounds.

In an in vitro test, selected compounds were tested for inhibition of polymerization of bovine brain. In this test, tubulin purified by cycles of polymerization and depolymerization, polymerization being initiated by addition of GTP and heating, is used. Table 1 states the IC₅₀ values of the inhibition of tubulin polymerization. Vincristine and colchicine, as known tubulin inhibitors, are included as reference substances. Highly potent inhibitors which may be emphasized are, for example, D-70316 and D-81187 having IC₅₀ values of 0.81 and 0.39 μM.

Table 1 furthermore states the cyctotoxic and growth-inhibiting activities of the compounds, tested using the human cervical carcinoma cell line HeLa/KB. Here, some of the compounds show themselves to be highly cytotoxic active compounds. Emphasis may be given, for example, to D-64131, D-68144, D-70316 and D-81187.

TABLE 1
Inhibition of the tubulin polymerization and cytotoxic activity on the HeLa/KB cervical carcinoma
cell line for selected compounds. The cytotoxicity and inhibition of growth is indicated in IC₅₀ in
the concentration μg/ml or nM.

| | | | Cytotoxicity |
|---|---|---|---|
| | | Tubulin inhibition/ | KB/HeLa |
| Example | Structure | IC50 [μM] | IC50 [μg/ml] |
| D-65499 | [See original for | 4.3(2) | ˜0.3 |
| (Ex. 115) | | | |
| D-65500 | structures here.] | 4.98(2) | ˜1.0 |
| (Ex. 114) | | | |
| D-65502 | | 5.58(2) | ˜0.5 |
| (Ex. 170) | | | |
| D-68887 (Bsp. 122) | 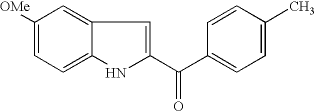 | 1.5(2) | ˜0.3 |
| D-68888 (Bsp. 108) | 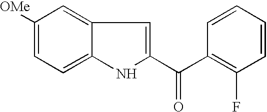 | 1.03(2) | ˜0.2 |
| D-68901 (Bsp. 111) | 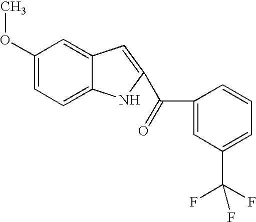 | 1.46(2) | ˜0.01 |
| D-68906 (Bsp. 126) | 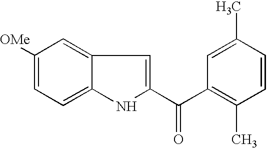 | 5.19(2) | ˜0.2 |
| D-70026 (Bsp. 113) | 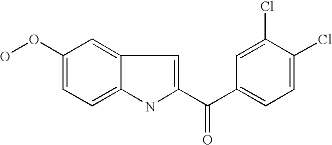 | 2.49(2) | ˜0.1 |
| D-70038 (Bsp. 110) | 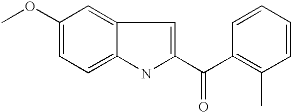 | 4.84(2) | ˜0.5 |
| D-70046 | 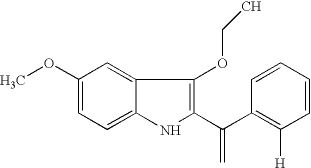 | 2.44(2) | ˜2 |
| D-70047 (Bsp. 128) | 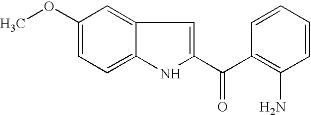 | 6.68(2) | ˜2 |
| D-70048 (Bsp. 129) | 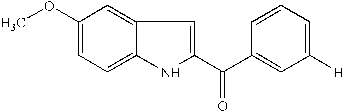 | 0.85(2) | ˜0.03 |
| D-70261 (Bsp. 103) | 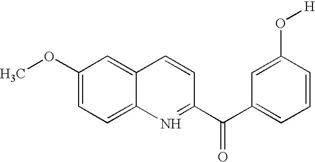 | 3.2(2) | n.b. |
| D-70288 (Bsp. 100) | 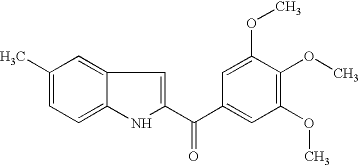 | 0.86(2) | ˜0.01 |
| D-70289 (Bsp. 97) | 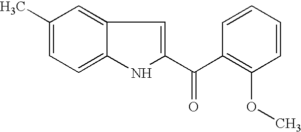 | 3.54(2) | ˜0.1 |
| D-70316 (Bsp. 99B) | 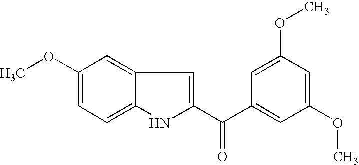 | 0.81(2) | 18.6 nM (2) |
| D-70438 | 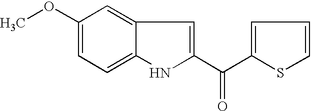 | 2.7(2) | n.b. |
| D-81167 (Bsp. 130) | 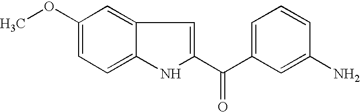 | 0.99(2) | 0.089 |
| D-81187 | 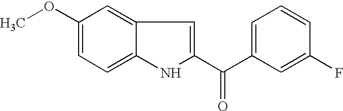 | 0.39(2) | 19.7 nM (2) |
| D-81194 | 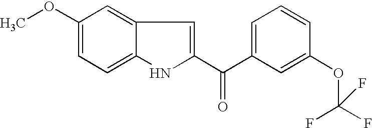 | 1.74(2) | 0.086 |
| D-81196 | 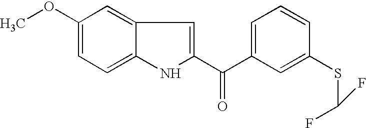 | 2.05(2) | 0.134 |
| D-81755 | 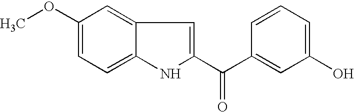 | 0.66(2) | 0.063 |
| D-81756 | 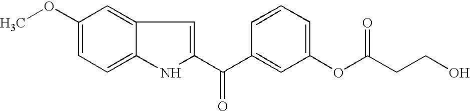 | 0.85(2) | 0.093 |
| Vincristin | 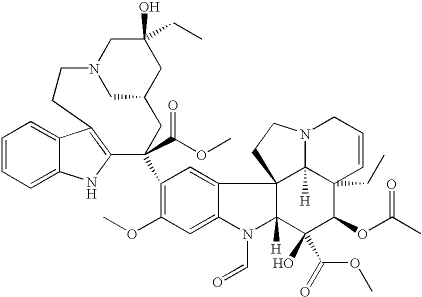 | 0.09(3) | 1.5 nM (2) |
| Colchicin | 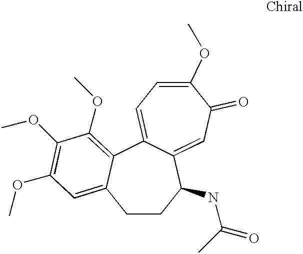 | 1.0(2) | 18.7 nM (2) |

### EXAMPLE 4

### Cell Cycle-Specific Action in the RKOp21 Model

The model used for examining the cell cycle-specific action was the RKOp21 cell system (M. Schmidt et al. Oncogene19(20):2423-9, 2000). RKO is a human colon carcinoma line in which the cell cycle inhibitor p21^{waf1} is expressed induced by the ecdysone expression system and leads to an arrest of the cell cycle specifically in G1 and G2. Nonspecific substances inhibit proliferation independently of whether the RKO cell is arrested in G1 or G2 or not. In contrast, cell cycle-specific substances, such as, for example, tubulin inhibitors, are only cytotoxic if the cells are not arrested and the cell cycle is in progress. Table 2 shows the cytotoxic and/or growth-inhibiting activity of selected compounds with/without expression of p21^{waf1}. All compounds tested showed little or no cytotoxic activity when p21^{waf1} was induced. This underlines the arrest of the cell cycle in Gl/M already found in the FACS analyses, and a cell cycle-specific action of the compounds tested.

TABLE 2
Cytotoxic activity of selected compounds in the RKO
p21^{waf1} cell system.

| | | RKP p21 induced |
|---|---|---|
| Substance | RKO p21 induced | IC₅₀ [nM] |
| D-64131 | n.d. | 15 (1) |
| D-68143 | n.d. | 28 (1) |
| D-68144 | n.d. | 3.6 (1) |
| D-68150 | n.d. | 16.8 (1) |
| D-68172 | n.d. | 136 (1) |
| D-70316 | n.d. | 17.95 (2) |
| D-81187 | n.d. | 16.8 (2) |
| Taxol | n.d. | 0.0078 μg/ml |

n.d. The IC₅₀ could not be determined

### EXAMPLE 5

### Activity of D-64131 in Human Tumor Xenograft Experiments in Naked Mice

For the in vivo experiments on naked mice, subcutaneously transplanted tumor fragments of the human melanoma MEXF 989 and/or the rhabdomyosarcoma SXF 463 were used. D-64131 was administered orally in doses of 100 and 200 mg/kg (vehicle 10% DMSO in PBS/Tween 80 0.05%) for 2 weeks (5 administrations per week; Monday to Friday). In experiments with both tumors, D-64131 was found to be highly effective. In the model MEXF989, it was possible to obtain a growth inhibition of 81% (200 mg/kg/day) or 66% (100 mg/kg/day) In the model SXF463, the higher dose of 200 mg/kg was found to cause 83% growth inhibition relative to the control. In addition to oral bioavailability and very good compatibilities, these results show potent anti-tumor activity in two human tumor xenograft models.

### Description of the Methods Used

Bovine Tubulin Polymerization Assay

The tubulin used in the assay was isolated from bovine brain using cycles of polymerization and depolymerization. 85 μl of a mixture comprising 80 μl of PEM buffer pH 6.6 (0.1M Pipes, 1 mM EGTA, 1 mM MgSO₄ p.H6.6) and 5 μl of 20 mM GTP stock solution per well were initially charged in the MultiScreen-type filter plate (0.22 μM hydrophilic, low protein binding-Durapore membrane, Millipore). The appropriate amount of test substance, dissolved in 100% DMSO, is pipetted to this mixture. 10 μl of purified bovine tubulin (50-60 μg of tubulin per well) are then added. The filter plate is shaken at room temperature and at 400 rpm for 20 min, and 50 μl/well of staining solution (45% MeOH, 10% acetic acid, 0.1% Naphthol Blue Black/Sigma) are then added using a pipette. Following an incubation time of 2 minutes, the staining solution is filtered off with suction (Eppendorf Event 4160), and the wells are then washed twice using a 90% methanol/2% acetic acid solution. 200 μl/well of decolorizing solution (25 mM NaOH, 50% ethanol, 0.05 mM EDTA) are finally added using a pipette. Following a 20 minute incubation at room temperature on a shaker (400 rpm), the absorption of 600 nM [sic] is measured in a photometer. What is calculated is the inhibition in % based on the 100% value of a positive control (which contains no test substance), and/or, if a concentration/activity curve is plotted, the IC₅₀ value.

XTT Test for Cellular Dehydroqenase Activity

In addition to the tumor cell line HeLa/KB (see Table 1), the cell lines C6, A-172, U118, U373, SKOV3 (ATCC HTB 77, human ovary adenocarcinoma), SF268 (NCI 503138, human glioma), NCI460 (NCI 503473; human non-small-cell lung carcinoma), MCF-7 (ATCC HTB22; human mamma adenocarcinoma) and RKO (human colon adenocarcinoma) can be used for the proliferation experiments.

Cell Cycle Analysis Using the RKOp21 Model

The assay is carried out in 96-well plates. By inducible expression of p21^{waf1}, the growth of the cells is arrested completely; however, the cells do not die. Comparison of the activity on induced and non-induced cells allows conclusions to be drawn about the mechanism of action (cell cycle specificity) of the therapeutics. Non-induced [sic] cells are sown at a cell count which is about four times higher than that of non-induced cells since, compared to non-induced cells, there is no further division during the assay (2×10⁴ cells/well induced, about 0.6×10⁴ cells/well not induced). The controls are untreated cells (+/− induction). Induction is carried out using 3 μM of muristerone A. On day 1, the cells are plated (+/− muristerone A) and incubated at 37° C. for 24 h. On day 2, the test substance is added (control DMSO) and incubation at 37° C. is continued for another 48 h, after which a standard XTT assay is carried out

Oral Bioavailability of D-64131:

Initially, D-64131 was examined in vitro for its antitumor activity using 12 permanent human tumor cell lines. The cell lines included intestinal (2), stomach (1), lung (3), breast (2), melanoma (2), ovary (1), kidney (1) and uterus (1) tumor cell lines. Using a propidium iodide-based cytotoxicity assay, the average IC50 of D-64131 for all cell lines examined was found to be 0.34 μM. Here, melanoma, intestinal and kidney tumor cells were most sensitive (IC50=4 nM). For the lung and stomach tumor cell lines, the IC50 was about 4 μM. Here, D-64131 acted as a cell cycle-specific active compound by interacting with tubulin. D-64131 inhibited polymerization of calf brain tubulin with an IC50 of 2.2 μM. For naked mice, the tolerated maximum dose in the case of intraperitoneal (i.p.) injection was 400 mg/kg when administered weekly. Peroral (p.o.) administration was carried out by administration of 100 and 200 mg/kg of D-64131, at the dosage “Qd×5” (1× daily for 5 successive days) for 2 weeks. Both p.o. dosages were tolerated very well and showed no indications of toxicity or loss of body weight. The latter dosage scheme was used for testing the activity of D-64131 in the human melanoma xenograft model MEXF 989. Oral treatment with D-64131 resulted in 81% growth inhibition compared to control at 200 mg/kg/d and 66% growth inhibition at 100 mg/kg/d. In the rhabdomyosarcoma xenograft model SXF 463, 83% growth inhibition at 200 mg/kg/d was found. The data found demonstrate that the indole compounds according to the invention are potent cytotoxically active compounds which act in a cell cycle-specific manner by interfering with the mitotic spindle apparatus. Emphasis is furthermore to be given to the oral bioavailability of the indole compounds according to the invention. Based on the observed activity and compatibility of the low-molecular-weight tubulin inhibitor D-64131, which is orally bioavailable, this compound is a candidate for further clinical phase I and II studies.

Examples of pharmaceutical preparations of the indole compounds according to the invention and their preparation are listed below.

### EXAMPLE I

### Tablet Containing 50 mg of Active Compound

Composition:

| | |
|---|---|
| (1) Active compound | 50.0 mg |
| (2) Lactose | 98.0 mg |
| (3) Corn starch | 50.0 mg |
| (4) polyvinylpyrrolidone | 15.0 mg |
| (5) Magnesium stearate | 2.0 mg |
| Sum: | 215.0 mg |

Preparation:

(1), (2) and (3) are mixed and granulated with an aqueous solution of (4). The dried granules are admixed with (5). This mixture is tabletted.

### EXAMPLE II

### Capsule Containing 50 mg of Active Compound

Composition:

| | |
|---|---|
| (1) Active compound | 50.0 mg |
| (2) Corn starch, dried | 58.0 mg |
| (3) Lactose powder | 50.0 mg |
| (4) Magnesium stearate | 2.0 mg |
| Sum: | 160.0 mg |

Preparation:

(1) is ground with (3). This ground material is added with vigorous mixing to the mixture of (2) and (4). This powder mixture is, on a capsule filling machine, filled into hard gelatin capsules size 3.

1. Use of at least one compound of the formula I 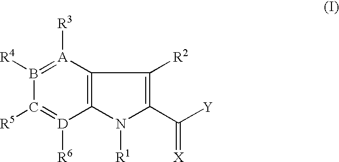
in which
R1 is hydrogen, (C1-C6)-alkylcarbonyl, preferably acetyl, (C₁-C₆)-alkyl, mono-(C1-C6)-alkylamino-(C₁-C₄)-alkyl, di-(C₁-C₆)-alkylamino-(C₁-C₄)-alkyl, where the two (C₁-C₆)-alkyl radicals together may form a ring, which optionally contains one or more NH, N-(C1-C6)-alkyl, O or S members, (C6-C14)-aryl-(C1-C6)-alkyl or (C6-C14)-aryl-(C1-C6)-alkoxy-(C1-C6)-alkyl;
R2 is a hydrogen atom, halogen, cyano, nitro, (C1-C6)-alkyl, (C1-C6)-alkyl which is substituted by one or more halogen atoms, preferably trifluoromethyl, (C1-C6)-alkoxy which is substituted by one or more halogen atoms, preferably trifluoromethoxy, (C2-C6)-alkenyl, (C2-C6)-alkynyl, (C3-C8)-cycloalkyl, (C1-C6)-alkoxy, (C1-C6)-alkoxycarbonyloxy, (C1-C6)-alkylcarbonyloxy, (C1-C4)-alkylthio, (C1-C4)-alkylsulfinyl, (C1-C4)-alkylsulfonyl, (C1-C6)-alkoxy-(C1-C6)-alkyl, amino, mono-(C1-C6)-alkylamino, di-N,N-(C1-C6)-alkylamino, where the two (C1-C6)-alkyl radicals together may form a ring, which optionally contains one or more NH, N-(C1-C6)-alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkylcarbonyl, (C1-C6)-alkoxycarbonyl or hydroxyl;
A, B, C and D independently of one another are a nitrogen atom (in which case R3, R4, R5 and R6 represent the free electron pair at the nitrogen atom) or are a carbon atom substituted by one of the radicals R3-R6;
R3, R4, R5 and R6 independently of one another are, when attached to nitrogen, a free electron pair, or, when attached to carbon, hydrogen, halogen, cyano, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, (C2-C6)-alkenyl, (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, (C1-C6)-alkoxycarbonyloxy, (C1-C6)-alkylcarbonyloxy, (C1-C4)-alkylthio, (C1-C4)-alkylsulfinyl, (C1-C4)-alkylsulfonyl, carboxyl, (C1-C6)-alkyl carboxylate, carboxamide, N-(C1-C4)-alkyl-carboxamide, N,N-di-(C1-C4)-alkyl-carboxamide, (C1-C6)-alkoxy-(C1-C6)-alkyl, amino, mono-(C1-C6)-alkylamino, N,N-di-(C1-C6)-alkyl-amino, where the two C1-C6-alkyl radicals together may form a ring, which optionally contains one or more NH, N-(C1-C6)-alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkylcarbonyl, (C1-C6)-alkylcarbonyloxy, (C1-C6)-alkoxycarbonyl, hydroxyl, where two directly adjacent radicals may be attached to one another;
Y is unsubstituted (C6-C14)-aryl or (C6-C14)-aryl which is fully or partially substituted by identical or different substituents, preferably phenyl or 1- or 2-naphthyl, or is unsubstituted (C1-C13)-heteroaryl or (C1-C13)-heteroaryl which is fully or partially substituted by identical or different substituents and has in each case at least one to four N, NH, N-(C1-C6)-alkyl, O and/or S as ring members, or is unsubstituted (C3-C8)-cycloalkyl or (C3-C8)-cycloalkyl which is fully or partially substituted by identical or different substituents, where the identical or different substituents are selected independently of one another from the group consisting of halogen, preferably fluorine, chlorine, bromine or iodine; cyano; straight-chain or branched cyano-(C1-C6)-alkyl; hydroxyl; straight-chain or branched (C1-C6)-alkyl which is substituted by one or more hydroxyl groups; carboxyl; (C1-C6)-alkyl carboxylate, carboxamide; N-(C1-C6)-alkyl-carboxamide, N,N-di-(C1-C4)-alkyl-carboxamide, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, straight-chain or branched (C2-C6)-alkenyl, straight-chain or branched (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, thio (—SH), straight-chain or branched (C1-C6)-alkylthio, (C1-C6)-alkylsulfinyl, (C1-C6)-alkylsulfonyl, (C1-C6)-alkoxy-(C1-C6)-alkyl, amino, straight-chain or branched mono-(C1-C6)-alkylamino, straight-chain or branched N,N-di-(C1-C6)-alkylamino, where the two (C1-C6)-alkyl radicals together may form a ring, which may optionally contain one or more NH, N-(C1-C6)-alkyl, O and/or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkylcarbonyloxy, (C1-C6)-alkoxycarbonyl, (C1-C6)-alkoxycarbonyloxy, straight-chain or branched mono- and N,N-di-(C1-C6)-alkylcarbonylamino, straight-chain or branched mono- and N,N-di-(C1-C6)-alkoxycarbonylamino, straight-chain or branched N-(C1-C6)-alkylcarbonyl-N-(C1-C6)-alkylamino, straight-chain or branched N-(C1-C6)-alkoxycarbonyl-N-(C1-C6)-alkylamino, formylamino, formyl, where two directly adjacent radicals may be attached to one another;
X is an oxygen or sulfur atom, is NH, or is a generally (at the same C atom) substituted hydroxyl and hydrogen (—CH(OH)—);
its stereoisomers, its tautomers, mixtures thereof and the pharmaceutically acceptable salts thereof, for preparing a medicament for the treatment of oncoses in mammals.

2. Use of at least one compound of the formula I according to claim 1, characterized in that R1-R6, A, B, C, D, X and Y are as defined in claim 1, with the proviso that at least one of the radicals R3-R6 is straight-chain or branched (C1-C6)-alkoxy, preferably methoxy; straight-chain or branched (C1-C6)-alkyl, preferably methyl; straight-chain or branched (C1-C6)-alkylenedioxy, preferably methylenedioxy, hydroxyl; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

3. Use of at least one compound of the formula I according to claim 1, characterized in that that [sic] R1, R2, R3, R5, R6, A, B, C, D, X and Y are as defined above, with the proviso that the radical R4 is straight-chain or branched (C1-C6)-alkoxy, preferably methoxy; straight-chain or branched (C1-C6)-alkyl, preferably methyl; straight-chain or branched (C1-C6)-alkylenedioxy (where the second oxygen atom may optionally be the radical R4 or R6), preferably methylenedioxy, hydroxyl; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

4. Use of at least one compound of the formula I according to claim 1, characterized in that that [sic] R1, R2, R3, R5, R6, A, B, C, D, X and Y are as defined above, with the proviso that the radical R4 is straight-chain or branched (C1-C6)-alkoxy, preferably methoxy.

5. Use of at least one compound of the formula I according to any of the preceding claims, characterized in that R1, R2, R3, R5, R6, A, B, C, D, X and Y are as defined above, with the proviso that the radical R4 is methoxy.

6. Use of at least one compound of the formula I according to any of the preceding claims, characterized in that R1-R6, A, B, C, D and X are as defined above, with the proviso that, the radical Y is substituted or unsubstituted (C6-C14)-aryl or (C1-C13)-heteroaryl which contains at least one to four N, NH, O and/or S as ring members.

7. Use of at least one compound of the formula I according to any of the preceding claims, characterized in that R1-R6, A, B, C, D and X are as defined above, with the proviso that the radical Y is (C6-C14)-aryl or (C1-C13)-heteroaryl which contains at least one N, NH, O and/or S as ring members, which is substituted by at least one radical selected from the group consisting of hydrogen, amino, halogen, nitro, cyano, straight-chain or branched (C1-C6)-alkoxy, preferably methoxy; straight-chain or branched (C1-C6)-alkyl, preferably methyl; hydroxyl; (C1-C6)-alkylcarbonyloxy, (C1-C6)-alkoxycarbonyloxy; straight-chain or branched (C1-C6)-alkoxy which is substituted by one or more halogen atoms, preferably trifluoromethoxy; straight-chain or branched (C1-C6)-alkyl which is substituted by one or more halogen atoms, preferably trifluoromethyl.

8. Use of at least one compound of the formula I according to any of the preceding claims, characterized in that R1-R6, A, B, C, D and X are as defined above, characterized in that the radical Y is a 1-phenyl radical which is unsubstituted or substituted by hydrogen, 3,4-dichloro, 2- or 3-methoxy, 2,4-dimethoxy, 3-nitro[lacuna]3-trifluoromethyl, 2,3,4-trimethoxy, 3,4,5-trimethoxy.

9. Use of a compound of the formula I according to any of claims 1 to 8 for preparing a medicament having antimitotic action in mammals.

10. Use of a compound of the formula I according to any of claims 1 to 8 for preparing a medicament for direct and/or indirect inhibition of tubulin polymerization in mammalian cells.

11. Compounds of the formula I 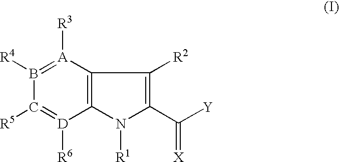
in which
R1 is hydrogen, (C1-C6)-alkylcarbonyl, preferably acetyl, (C₁-C₆)-alkyl, mono-(C₁-C₆)-alkylamino-(C₁-C₄)-alkyl, di(C₁-C₆)-amino-(C₁-C4)-alkyl, where the two (C₁-C4)-alkyl radicals together may form a ring, which optionally contains one or more NH, N-(C1-C[lacuna])-alkyl, O or S members, (C6-C14)-aryl-(C1-C6)-alkyl or (C6-C14)-aryl-(C1-C6)-alkoxy-(C1-C6)-alkyl;
R2 is a hydrogen atom, halogen, cyano, nitro, (C1-C6)-alkyl, (C1-C6)-alkyl which is substituted by one or more halogen atoms, preferably trifluoromethyl, (C1-C6)-alkoxy which is substituted by one or more halogen atoms, preferably trifluoromethoxy, (C2-C6)-alkenyl, (C2-C6)-alkynyl, (C3-C8)-cycloalkyl, (C1-C[lacuna])-alkoxy, (C1-C[lacuna])-alkoxycarbonyloxy, (C1-C[lacuna])-alkylcarbonyloxy, (C1-C)-alkylthio, (C1-C4)-alkylsulfinyl, (C1-C4)-alkylsulfonyl, (C1-C4)-alkoxy-(C1-C4)-alkyl, amino, mono-(C1-C[lacuna])-alkylamino, di-(C1-C[lacuna])-alkyl)-amino, where the two C1-C4-alkyl radicals together may form a ring, which optionally contains one or more NH, N-(C1-C4)alkyl, O or S, (C6-C14)-aryl, (C6-C14)-aryloxy, (C6-C14)-aryl-(C1-C4)-alkyl, (C6-C14)-aryl-(C1-C4)-alkoxy-(C1-C4)-alkyl, (C1-C6)-alkyl-carbonyl, (C1-C6)-alkoxycarbonyl or hydroxyl;
A, B, C and D independently of one another are a nitrogen atom (in which case R3, R4, R5 and R6 represent the free electron pair at the nitrogen atom) or are a carbon atom substituted by one of the radicals R3-R6;
R3, R4, R5 and R6 independently of one another are, when attached to nitrogen, a free electron pair, or, when attached to carbon, hydrogen, halogen, cyano, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, (C2-C6)-alkenyl, (C2-C6)-alkynyl, (C3-C8)-cycloalkyl, straight-chain or branched (C1-C6)-alkoxy, straight-chain or branched (C1-C6)-alkylenedioxy, (C1-C6)-alkoxy-carbonyloxy, (C1-C6)-alkylcarbonyloxy, (C1-C[lacuna])-alkylthio, (C1-C4)-alkylsulfinyl, (C1-C4)-alkylsulfonyl, carboxyl, (C1-C6)-alkyl carboxylate, carboxamide, N-(C1-C4)-alkyl-carboxamide, N,N-di-(C1-C4)-alkyl-carboxamide, (C1-C6)-alkoxy-(C1-C6)-alkyl, amino, mono-(C1-C6)-alkylamino, di-(C1-C6)-alkyl)-amino, where the two C1-C4-alkyl radicals together may form a ring, which optionally contains one or more NH, N-(C1-C4)-alkyl, O or S, aryl, aryloxy, aryl-(C1-C4)-alkyl, aryl-(C1-C4)-alkoxy-(C1-C4)-alkyl, (C1-C6)-alkylcarbonyl, (C1-C6)-alkoxycarbonyl, hydroxyl, where two directly adjacent radicals may be attached to one another;
Y is unsubstituted (C10-C14)-aryl or (C10-C14)-aryl which is fully or partially substituted by identical or different substituents, preferably 1- or 2-naphthyl, or is unsubstituted (C1-C13)-heteroaryl or (C1-C13)-heteroaryl which is fully or partially substituted by identical or different substituents and has in each case at least one to four N, NH, N-(C1-C6)-alkyl, O and/or S as ring members, or is unsubstituted (C3-C8)-cycloalkyl or (C3-C8)-cycloalkyl which is fully or partially substituted by identical or different substituents, where the identical or different substituents are selected independently of one another from the group consisting of halogen, preferably fluorine, chlorine, bromine or iodine; cyano; straight-chain or branched cyano-(C1-C6)-alkyl; hydroxyl; straight-chain or branched (C1-C6)-alkyl which is substituted by one or more hydroxyl substituents; carboxyl; (C1-C6)-alkyl carboxylate; carboxamide; N-(C1-C6)-alkyl-carboxamide, N,N-di-(C1-C4)-alkyl-carboxamide, nitro, straight-chain or branched (C1-C6)-alkyl, straight-chain or branched (C1-C6)-alkyl which is substituted by one or more halogen atoms, preferably trifluoromethyl, straight-chain or branched (C1-C6)-alkoxy which is substituted by one or more halogen atoms, preferably trifluoromethoxy, straight-chain or branched (C2-C6)-alkenyl, straight-chain or branched (C2-C6)-alkynyl, (C3-C8)-cycloalkyl, straight-chain or branched (C1-C6)-alkoxy, preferably methoxy, straight-chain or branched (C1-C6)-alkylenedioxy, preferably methylenedioxy, thio (—SH), straight-chain or branched (C1-C6)-alkylthio, (C1-C6)-alkylsulfinyl, (C1-C6)-alkylsulfonyl, (C1-C6)-alkoxy-(C1-C6)-alkyl, amino, straight-chain or branched mono-(C1-C6)-alkylamino, straight-chain or branched N,N-di-(C1-C6)-alkylamino, where the two (C1-C6)-alkyl radicals together may form a ring, which may optionally contain one or more NH, N-(C1-C6)-alkyl, O and/or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkylcarbonyloxy, (C1-C6)-alkoxycarbonyl, (C1-C6)-alkoxycarbonyloxy, straight-chain or branched mono- and N,N-di-(C1-C6)-alkylcarbonylamino, straight-chain or branched mono-N- and N,N-di-(C1-C6)-alkoxycarbonylamino, straight-chain or branched N-(C1-C6)-alkylcarbonyl-N-(C1-C6)-alkylamino, straight-chain or branched N-(C1-C6)-alkoxycarbonyl-N-(C1-C6)-alkylamino, formylamino, formyl, where two directly adjacent radicals may be attached to one another;
X is an oxygen or sulfur atom, is NH, or is a generally (at the same C atom) substituted hydroxyl and hydrogen (—CH(OH)—);
their stereoisomers, their tautomers, and the pharmaceutically acceptable salts thereof, except for the racemic compounds according to formula I where R1=R2=R3=R5=R6=hydrogen, X=oxygen or, if R4=H, generally substituted hyroxyl and hydrogen, Y=3-carboxypyridin-4-yl and R4=hydrogen or methoxy, and the compounds 2-cyclopropylcarbonylindole and 2-cyclohexyl-carbonylindole.

12. Compounds of the formula I according to claim 11 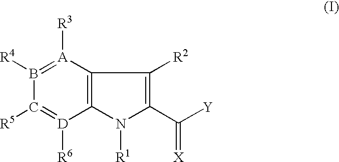
in which A, B, C, D, X, Y and R1 to R6 are as defined in claim 13 [sic], including the compounds of the formula I where R1=R2=R3=R5=R6=hydrogen, X=oxygen or, if R4=H, generally substituted hydroxyl and hydrogen, Y×3-carboxypyridin-4-yl and R4=hydrogen or methoxy, and the compounds 2-cyclopropyl-carbonylindole and 2-cyclohexylcarbonylindole for use as medicaments, in particular as antitumor agents.

13. Antitumor agent, comprising an effective amount of at least one compound of the formula according to any of claims 1 to 10, if appropriate together with customary pharmaceutical auxiliaries or excipients.

14. Medicament, in particular antitumor agent, comprising an effective amount of at least one compound of the formula as claimed in claims 11 or 12, if appropriate together with customary pharmaceutical auxiliaries and/or excipients.

15. Medicament according to claim 13 or 14, characterized in that it can be administered orally, perorally or topically to a mammal.
